(19) United States
(12) Patent Application Publication
(10) Pub. No.: US 2024/0307397 A1
(43) Pub. Date: Sep. 19, 2024

(54) PHARMACEUTICAL COMPOSITION COMPRISING PI3K AND DNA-PK DUAL INHIBITOR FOR PREVENTING OR TREATING PERIPHERAL T CELL LYMPHOMA

(71) Applicant: BORYUNG CORPORATION, Seoul (KR)

(72) Inventors: Bong-Seog KIM, Seoul (KR); Hyo Soon JUNG, Seoul (KR); Kyoung Ryun PARK, Seongnam-si, Gyeonggi-do (KR); Seungho WANG, Siheung-si, Gyeonggi-do (KR); Seung Hee JUNG, Seoul (KR); Yoon Sik KIM, Suwon-si, Gyeonggi-do (KR); Joo Han LEE, Seoul (KR); Byeongwook JEON, Incheon (KR); Jongyeon KIM, Suwon-si, Gyeonggi-do (KR); Tae Min KIM, Seoul (KR); Seok Jin KIM, Seoul (KR); Dok Hyun YOON, Seoul (KR); Yoon Kyung JEON, Seoul (KR); Ahmad H. MATTOUR, Livonia, MI (US); Jorge M. CHAVES, Tacoma, WA (US)

(21) Appl. No.: 18/654,486

(22) Filed: May 3, 2024

Related U.S. Application Data
(63) Continuation of application No. PCT/KR2022/017128, filed on Nov. 3, 2022.

(30) Foreign Application Priority Data
Nov. 3, 2021 (KR) 10-2021-0150125

(51) Int. Cl. A61K 31/519; A61K 9/00; A61P 35/00

(52) U.S. Cl. CPC A61K 31/519; A61K 9/0053; A61P 35/00

(57) ABSTRACT

A composition for preventing or treating peripheral T-cell lymphoma includes a compound represented by formula 1 or pharmaceutically acceptable salts thereof. Specifically, a pharmaceutical composition based on the composition is capable of effectively ameliorating peripheral T-cell lymphoma by decreasing regulatory T cells and increasing CD8⁺ T cells. The pharmaceutical composition may exhibit anticancer activity against peripheral T-cell lymphoma by decreasing regulatory T cells which suppress immune responses and increasing CD8⁺ T cells which exhibit anticancer activity. In addition, the pharmaceutical composition is used for the treatment of peripheral T-cell lymphoma due to excellent safety and tolerability.

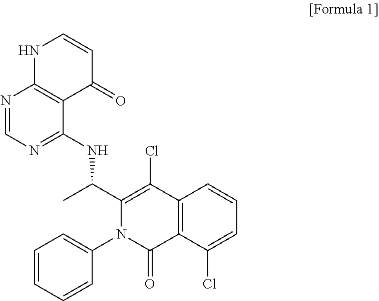 [SMILES: C[C@H](Nc1ncnc2[nH]ccc(=O)c12)c1c(Cl)c2cccc(Cl)c2c(=O)n1-c1ccccc1]

### CROSS-REFERENCE TO RELATED APPLICATIONS

This application is a Continuation of International Application No. PCT/KR2022/017128 filed Nov. 3, 2022, which claims priority from Korean Application No. 10-2021-0150125 filed Nov. 3, 2021. The aforementioned applications are incorporated herein by reference in their entireties.

### TECHNICAL FIELD

The present invention relates to a composition for preventing or treating peripheral T-cell lymphoma, including a compound represented by formula 1 or pharmaceutically acceptable salts thereof. Specifically, the present invention relates to a pharmaceutical composition capable of effectively ameliorating peripheral T-cell lymphoma by decreasing regulatory T cells and increasing CD8⁺ T cells.

### RELATED ART

Phosphatidylinositol 3-kinase (PI3 kinase; PI3K) is a lipid kinase, which phosphorylates lipid molecules instead of proteins, and plays an important role in cell survival, signal transduction, control of membrane trafficking, etc. If a problem occurs to such control of cell survival, signal transduction, control of membrane trafficking, cancer, inflammatory diseases, autoimmune diseases, etc. occur.

Recently, the results of a study on developing a compound having a novel structure effective in selectively inhibiting PI3 kinase have been reported, and specifically International Publication WO 2016/204429 discloses a compound which has PI3K inhibitory activity and is useful for the treatment of cancer, autoimmune diseases, respiratory diseases and the like, which is incorporated herein by reference. However, a PI3K-δ inhibitor monotherapy is known to have a poor prognosis in non-Hodgkin's lymphoma, which is a malignant lymphoma.

Meanwhile, inhibitors for suppressing multiple PI3K subtypes and/or DNA-PK activation other than PI3K-δ only inhibitors efficiently induce cell cycle arrest and apoptosis, stimulate a decrease in the stability of carcinogens, and thus are expected to exhibit effective anticancer activity in indolent non-Hodgkin's lymphoma, too.

Currently, it is known that about 4,300 cases of non-Hodgkin's lymphoma occur in Korea and approximately 510,000 cases worldwide each year, but therapeutic agents for non-Hodgkin's lymphoma involving multiple inhibitors for suppressing multiple PI3K subtypes and/or DNA-PK activation are not known, and thus there is a need for developing the therapeutic agents.

### RELATED ART REFERENCES

### Patent Documents

International Publication WO 2016/204429

### SUMMARY

An object of the present invention is to provide a pharmaceutical composition for preventing or treating peripheral T-cell lymphoma (PTCL), including a compound represented by formula 1 below or pharmaceutically acceptable salts thereof in an amount of 25 mg or more and less than 325 mg as a compound represented by formula 1.

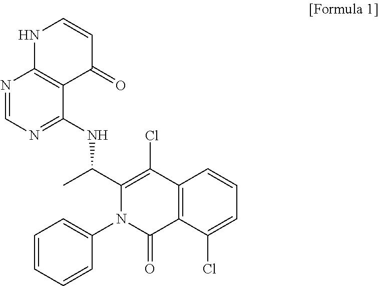 [SMILES: C[C@H](Nc1ncnc2[nH]ccc(=O)c12)c1c(Cl)c2cccc(Cl)c2c(=O)n1-c1ccccc1]

The present invention provides a pharmaceutical composition for preventing or treating peripheral T-cell lymphoma (PTCL), including a compound represented by formula 1 below or pharmaceutically acceptable salts thereof in an amount of 25 mg or more and less than 325 mg as the compound.

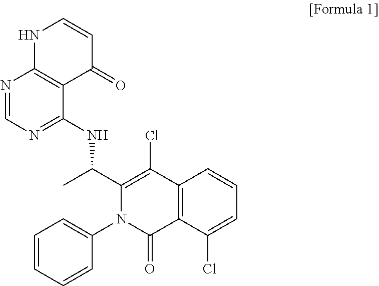 [SMILES: C[C@H](Nc1ncnc2[nH]ccc(=O)c12)c1c(Cl)c2cccc(Cl)c2c(=O)n1-c1ccccc1]

In the present invention, the pharmaceutical composition may be used for preventing or treating peripheral T-cell lymphoma (PTCL), relapsed/refractory B-cell lymphoma, chronic lymphocytic leukemia (CLL)/small lymphocytic leukemia (SLL) other than peripheral T-cell lymphoma (PTCL), and specifically may be used for preventing or treating diffuse large B-cell lymphoma (DLBCL), including MYC-altered diffuse large B-cell lymphoma and transformed diffuse large B-cell lymphoma; follicular lymphoma; chronic Lymphocytic Leukemia (CLL)/small lymphocytic leukemia (SLL); other B-cell lymphomas such as mantle cell lymphoma (MCL), marginal zone lymphoma (MZL), and Waldenstrom macroglobulinemia; and cutaneous T cell lymphoma/mycosis fungoide (composite CTCL-MF) without limitation.

The compound represented by above formula 1 is one of heteroaryl derivatives known to have PI3K and DNA-PK dual inhibitory activity, and is named “4-[[(1S)-1-(4,8-dichloro-1-oxo-2-phenyl-3-isoquinolyl)ethyl]amino]-8H-pyrido[2,3-d]pyrimidin-5-one.”

In the present invention, the pharmaceutically acceptable salts mean salts conventionally used in a pharmaceutical industry, for example, inorganic ion salts prepared from calcium, potassium, sodium, magnesium or the like; inorganic acid salts prepared from hydrochloric acid, nitric acid, phosphoric acid, bromic acid, iodic acid, perchloric acid, sulfuric acid or the like; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid or the like; sulfonic acid salts prepared from methanesulfonic acid, ethanesulfonic acid, benzenesulfonic acid, p-toluenesulfonic acid, naphthalenesulfonic acid or the like; amino acid salts prepared from glycine, arginine, lysine, etc.; amine salts prepared from trimethylamine, triethylamine, ammonia, pyridine, picoline, etc.; or the like, but types of salt meant in the present invention are not limited to those listed salts.

In the present invention, the composition may include the compound represented by above formula 1 or pharmaceutically acceptable salts thereof as the compound in an amount of 25 mg or more and less than 325 mg, 50 mg or more and less than 325 mg, 100 mg or more and less than 325 mg, or 200 mg or more and less than 325 mg, but is not limited thereto.

In one embodiment of the present invention, the composition may include the compound represented by above formula 1 or pharmaceutically acceptable salts thereof as the compound in an amount of 50 mg, 100 mg or 200 mg, preferably 200 mg.

When administering the composition to a subject with peripheral T-cell lymphoma (PTCL) according to the present invention, a maximum tolerated dose (MTD) of the compound represented by above formula 1 may be 200 mg QD, and have excellent tolerability and safety in this dose range. In addition, based on the tolerability and safety, 200 mg QD of the compound represented by above formula 1 may be recommended as a starting dose (RP2D) for clinical phase Ib/II.

The above dose range in the composition according to the present invention may have safety and tolerability in subjects with peripheral T-cell lymphoma (PTCL), and thus may be used for the treatment of subjects diagnosed with peripheral T-cell lymphoma (PTCL).

In the composition according to the present invention, the pharmaceutically acceptable salt of the compound represented by above formula 1 may mean the compound represented by above formula 1 which is included in an amount of 25 mg or more and less than 325 mg, 50 mg or more and less than 325 mg, 100 mg or more and less than 325 mg, and 200 mg or more and less than 325 mg but is not limited thereto, preferably 25 mg, 50 mg, 100 mg or 200 mg, and more preferably 200 mg.

The present invention may effectively prevent or treat peripheral T-cell lymphoma (PTCL) by showing excellent safety and tolerability in subjects with peripheral T-cell lymphoma (PTCL) in the above dose range.

In the present invention, the pharmaceutical composition may be administered to a subject in need thereof in an amount of 25 mg or more and less than 325 mg, 50 mg or more and less than 325 mg, 100 mg or more and less than 325 mg, 200 mg or more and less than 325 mg, preferably 25 mg, 50 mg, 100 mg or 200 mg once daily (QD) for 28 consecutive days (cycle 1, four weeks) or more.

For example, subjects who have completed the first cycle may continue to receive a test drug at the same dose as in cycle 1 until the occurrence of tumor progression, toxicity exceeding a tolerability limit, or withdrawal of consent. For example, the subjects may receive the test drug for at least 4, at least 8, at least 12, at least 16, at least 20, at least 24, at least 28, at least 32, or at least 36 weeks, but are not limited thereto.

In the present invention, if emerging new safety, tolerability, and PK data indicate that a more frequent dosing regimen would be appropriate, a dosing regimen such as once daily (QD) may be changed to twice daily (BID), but is not limited thereto. In addition, the dose of the compound represented by above formula 1 according to the present invention may be subject to change taking a total daily exposure of the new regimen (area under the concentration-time curve, AUC) into account, as long as the dose does not exceed the AUC established as safe and tolerated after QD administration.

In the present invention, the pharmaceutical composition may be administered to a subject diagnosed with peripheral T-cell lymphoma (PTCL), specifically, a subject who has received at least one chemotherapy after being diagnosed with peripheral T-cell lymphoma (PTCL). The chemotherapy may refer to a systemic treatment method which prevents cancer cells from growing or kills the same by using a drug (anticancer agent), which is a standard treatment method for cancer along with surgery and radiation therapy, and may include anthracycline-based chemotherapy (regimens), rituximab and/or nivolumab-based immunotherapy, but is not limited thereto.

In the present invention, the “subject” may mean mammals including humans, and specifically may mean humans.

In the present invention, the above pharmaceutical composition may be orally or parenterally administered (for example, intravenously, subcutaneously, intraperitoneally or locally administered), and a dose thereof may be appropriately changed depending on an administration route. In one embodiment of the present invention, the composition may be orally administered.

In the present invention, the term “oral administration” may mean one, in which a substance prepared for allowing an active substance to be digested is administered into the gastrointestinal tract for absorption. A non-limiting example of the preparation for oral administration may include tablets, troches, lozenges, water soluble suspensions, oil suspensions, prepared powders, granules, emulsions, hard capsules, soft capsules, syrups, elixirs or the like. To formulate the pharmaceutical composition of the present invention into a preparation for oral administration, the followings may be used: binders such as lactose, saccharose, sorbitol, mannitol, starch, amylopectin, cellulose, gelatin or the like; diluents such as dicalcium phosphate, etc.; disintegrants such as maize starch, sweet potato starch or the like; lubricants such as magnesium stearate, calcium stearate, sodium stearyl fumarate, polyethylene glycol wax, or the like; etc., in which sweetening agents, flavoring agents, syrups, etc. may also be used. Furthermore, in case of the capsules, liquid carriers such as fatty oil, etc. may be further used in addition to the above-mentioned materials.

The pharmaceutical composition may be prepared in the form of oral or parenteral preparation, which is apparent to those skilled in the art. In one embodiment of the present invention, the pharmaceutical composition may be a preparation for oral administration. In the present invention, the above preparation for oral administration may be a solid preparation such as a tablet, pill, powder, granule, capsule, etc. or a liquid preparation such as a suspension, liquid for internal use, emulsion, syrup, etc., preferably the solid preparation, more preferably the capsule.

If the pharmaceutical composition of the present invention is formulated into an oral solid preparation, the examples of the additives used herein may include cellulose, calcium silicate, corn starch, lactose, sucrose, dextrose, calcium phosphate, stearic acid, magnesium stearate, calcium stearate, gelatin, talc, etc.

If the pharmaceutical composition is formulated into a liquid preparation for oral administration, the pharmaceutical composition may be formulated by adding simple diluents such as water, liquid or paraffin, and various additives such as a wetting agent, sweetener, fragrance, preservative, antiseptic, colorant, etc.

When the pharmaceutical composition of the present invention is prepared in the form of an injection, the additives may include water, saline, aqueous glucose solution, pseudo-sugar aqueous solution, alcohol, glycol, ether (e.g., polyethylene glycol 400), oil, fatty acid, fatty acid ester, glyceride, etc.

In the present invention, the pharmaceutical composition may decrease regulatory T cells (T_{regs}) and increase CD8⁺ T cells. The pharmaceutical composition may exhibit excellent anticancer activity against advanced hematologic cancer by decreasing regulatory T cells which suppress immune responses and increasing CD8⁺ T cells which exhibit anticancer effects. In one embodiment of the present invention, the advanced hematologic cancer may be peripheral T-cell lymphoma (PTCL).

In the present invention, the pharmaceutical composition may further include an active ingredient which has pharmacological activity other than the compound represented by above formula 1 or pharmaceutically acceptable salts thereof.

In the present invention, the pharmaceutical composition may further include a pharmaceutically acceptable additive. As used herein, the “pharmaceutically acceptable” may mean one which is physiologically acceptable; which does not conventionally cause an allergic reaction such as gastrointestinal disturbance and dizziness, or other similar reactions thereto, when being administered into a human; and which is conventionally used by those skilled in the art in preparing a pharmaceutical composition with reference to Remington's Pharmaceutical Science (latest version), Mack Publishing Company, Easton PA.

The above additive may be a carrier, diluent, extender, antioxidant, buffer, filler, anti-coagulant, lubricant, humectant, a flavoring agent, emulsifier, a suspending agent, surfactant, antiseptic, etc. For example, the above additive may be lactose, dextrose, calcium silicate, maize starch, sucrose, sorbitol, mannitol, xylitol, erythritol, maltitol, starch, acacia rubber, alginate, gelatin, calcium phosphate, calcium silicate, cellulose, methyl cellulose, polyvinyl pyrrolidone, water, methyl hydroxybenzoate, propyl hydroxybenzoate, talc, stearic acid, magnesium stearate, calcium stearate, mineral oil, saline solution, glucose aqueous solution, similar sugar aqueous solution, alcohol, glycol, ether (ex: polyethylene glycol 400), oil, fatty acid, fatty acid ester, glyceride or mixtures thereof. However, additives which may be contained in the composition according to the present invention are not limited to those listed substances, which are intended as examples only. In the present invention, a content of additives contained in the pharmaceutical composition is not particularly limited, and may be appropriately adjusted within a range of contents conventionally used in formulating a preparation.

In the present invention, the term “prevention” may refer to an inhibition or a delay of occurrence of disease, disorder or condition. If the occurrence of disease, disorder or condition is inhibited or delayed for an expected period of time, the prevention may be considered as complete.

In the present invention, the term “treatment” may refer to one which partially or completely reduces, ameliorates, alleviates, inhibits or delays a certain disease, disorder and/or condition, or symptoms of the condition, reduces severity thereof, or reduces the occurrence of at least one symptom or property thereof.

The present invention may provide a use of a pharmaceutical composition for preventing or treating advanced hematologic cancer, which includes a compound represented by formula 1 below or pharmaceutically acceptable salts thereof in an amount of 25 mg or more and less than 325 mg as a compound represented by formula 1. In one embodiment of the present invention, the advanced hematologic cancer may be peripheral T-cell lymphoma (PTCL).

The present invention may provide a use of a pharmaceutical composition in the manufacture of a medication for preventing or treating advanced hematologic cancer, which includes a compound represented by formula 1 below or pharmaceutically acceptable salts thereof in an amount of 25 mg or more and less than 325 mg as a compound represented by formula 1. In one embodiment of the present invention, the advanced hematologic cancer may be peripheral T-cell lymphoma (PTCL).

The present invention may provide a method for preventing or treating advanced hematologic cancer by administering into a subject a pharmaceutical composition which includes a compound represented by formula 1 below or pharmaceutically acceptable salts thereof in an amount of 25 mg or more and less than 325 mg as a compound represented by formula 1. In one embodiment of the present invention, the advanced hematologic cancer may be peripheral T-cell lymphoma (PTCL).

Matters mentioned in the pharmaceutical composition of the present invention, the treatment method thereof and the use thereof are applied the same, if not contradictory to each other.

The pharmaceutical composition of the present invention may have excellent safety and tolerability in the treatment of peripheral T-cell lymphoma.

In addition, the pharmaceutical composition of the present invention may effectively treat peripheral T-cell lymphoma by decreasing regulatory T cells which suppress immune responses and by increasing CD8⁺ T cells which exhibit anticancer effects.

### BRIEF DESCRIPTION OF THE DRAWINGS

FIG. 1 is a schematic view showing a clinical trial phase I design.

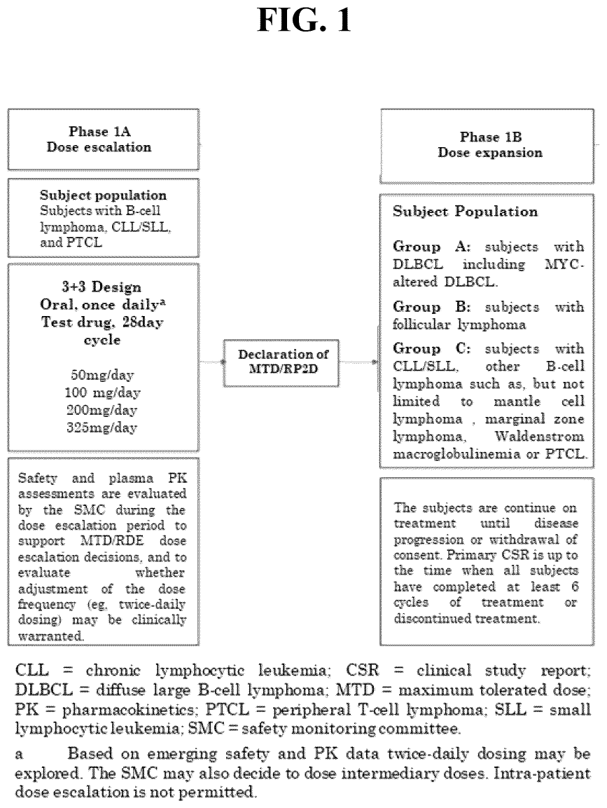

FIG. 2 is a schematic view showing a dose escalation trial design (cohorts 1 and 2).

FIG. 3 is a schematic view showing a dose escalation trial design (cohorts 3 and subsequent).

FIG. 4 shows the results of pharmacokinetic (PK) analyses of plasma samples on day 1 of cycle 1 (C1D1) after administration of a test drug to a subject.

FIG. 5 shows the results of pharmacokinetic (PK) analyses of plasma samples on day 15 of cycle 1 (C1D15) after administration of a test drug to a subject.

FIG. 6A is a scatter plot of a PK-PD correlation of changes in regulatory T cell expression after administration of a test drug.

FIG. 6B is a scatter plot of a PK-PD correlation of changes in CD8⁺ T cell expression after administration of a test drug.

### DETAILED DESCRIPTION

Hereinafter, the present invention will be described in detail through Examples for better understanding of the present invention. However, the following Examples are provided only for the purpose of illustrating the present invention, and thus the scope of the present invention is not limited thereto. The Examples of the present invention are provided to more completely describe the present invention to those having ordinary skill in the art.

### EXAMPLES

### 1. Subjects Inclusion

A total of 12 subjects with advanced hematologic malignancies were included for studying of phase Ia of this clinical trial.

The above subjects included those who have pathological subtypes of peripheral T-cell lymphoma (PTCL) (n=8), diffuse large B-cell lymphoma (DLBCL) (n=2), marginal zone lymphoma (MZL) (n=1), and cutaneous T-cell lymphoma/mycosis fungoides (composite CTCL-MF) (n=1).

The demographic and other underlying characteristics of the above subjects are as follows:
Male (n=9) and female (n=3)
Median age (63, range 30-76)
ECOG PS (0-1)
All the subjects have previously experienced at least one chemotherapy.

### A. Inclusion Criteria

The subjects are considered to be eligible for participating in the clinical trial only if they meet all of the following criteria:

1) Be capable of giving signed informed consent. This also includes complying with the requirements and restrictions listed in the informed consent form (ICF) and in the protocol.

2) Male or female subjects 18 years of age or older (If a legal age of consent to participate in clinical trials is >18 years old, local regulatory requirements need to be followed.)

3) ECOG performance status≤2.

4) Life expectancy in excess of 3 months.

5) Phase Ia (dose escalation part only):

Those who have received a standard therapy or are intolerant of standard therapy, and/or for whom no standard therapy exists, while being diagnosed with relapsed and/or refractory B-cell lymphoma, chronic lymphocytic leukemia (CLL)/small lymphocytic leukemia (SLL) and peripheral T-cell lymphoma (PTCL) according to the World Health Organization (WHO) classification.

6) Phase Ib (dose expansion only):

Those who have received a standard therapy or are intolerant of standard therapy, or for whom no standard therapy exists and fit into one of the following groups, while being diagnosed with relapsed and/or refractory lymphoma (according to the World Health Organization (WHO) classification).
Group A: Subjects who are diagnosed with diffuse large B-cell lymphoma (DCBCL), including MYC-altered DLBCL and transformed DLBCL.
Group B: Subjects who are diagnosed with grade 1 to 3a follicular lymphoma.
Group C: Subjects who are diagnosed with CLL/SLL and other B-cell lymphomas (e.g., including, but not limited to, mantle cell lymphoma (MCL), marginal zone lymphoma (MZL), Waldenstrom macroglobulinemia, or peripheral T-cell lymphoma (PTCL)).

7) All the subjects enrolled in phases la and Ib (dose expansion) should have measurable disease based on the appropriate tumor type criteria.

8) Subjects currently in need of systemic therapy according to an investigator's assessment.

9) An archival or fresh tumor tissue (ie, tissue block or series of approximately 15 slides) is required and should be provided during the screening visit for lymphoma subjects. For CLL and Waldenstrom macroglobulinemia, this is not required if no available tumor tissue.

10) the first 7 subjects in each group of Phase la and Phase Ib should be prepared to undergo a fresh tumor biopsy during a clinical trial period. For the CLL and Waldenstrom macroglobulinemia, the biopsy is not mandatory. For subjects with disease sites unsuitable for biopsy, exceptions may be considered after discussion with a sponsor.

11) Female subjects must be surgically sterile, or have a monogamous partner who is surgically sterile, or be of postmenopausal status (at least 1 year amenorrhea or serum follicle-stimulating hormone>40 mIU/mL and estradiol<20 pg/mL or a condition as defined to be in a “postmenopausal” range for related trials), or commit to use an effective form of birth control for the duration of clinical trial and for 3 months after the last test drug administration.

12) Sexually active males must commit to use an effective form of birth control during the period of taking the test drug and for 3 months after stopping the administration of the test drug. A condom is required to be used by vasectomized men to prevent delivery of the drug via seminal fluid.

13) Those who represent laboratory test values defined as follows:
Creatinine clearance (calculated using the Cockcroft-Gault formula, or measured)>60 mL/min.
Total bilirubin<1.5×ULN (except for subjects with Gilbert's syndrome who are excluded if total bilirubin>3.0×ULN or direct bilirubin<1.5×ULN).
ALT<2.5×ULN (except for subjects who have tumor involvement of the liver, who are included if ALT<5×ULN).
Absolute neutrophil count (ANC)>1.0×10⁹/L
Platelet count>75×10⁹/L
Hemoglobin>8 g/dL

### B. Exclusion Criteria

Subjects who meet any one of the following criteria are excluded from this clinical trial.

1) Presence of overt leptomeningeal or active central nervous system (CNS) metastases, or CNS metastases that require local CNS-directed therapy (e.g., radiotherapy or surgery) or increasing doses of corticosteroids within the prior 2 weeks. Brain metastasis subjects who received treatment need to be in a neurologically stable state (for four weeks after administration and before enrollment into clinical trial), and off steroids for at least two weeks before administration of the test drug.

2) Impaired cardiac function or clinically significant cardiac disease, including any of the following:
Clinically significant and/or uncontrolled heart disease such as congestive heart failure requiring treatment (New York Heart Association Grade≥2), left ventricular ejection fraction<50% as determined by multiple gated acquisition (MUGA) or echocardiogram (ECHO), uncontrolled hypertension, or clinically significant arrhythmia.
QT interval corrected for heart rate using Fridericia's formula (QTcF)>450 ms ECG or congenital long QT syndrome at the Screening Visit. If the QTcF cannot be calculated appropriately due to underlying conditions such as Left Bundle Branch Block (LBBB), the eligibility of the subject will be discussed with the medical monitor and Sponsor. The decision of eligibility will be documented appropriately.
A marked baseline prolongation of QT/QTc interval (e.g., repeated demonstration of a QTc interval>450 ms).
The obligatory use of concomitant medications that prolong the QT/QTc interval.
Acute myocardial infarction or unstable angina pectoris<3 months prior to clinical trial entry.

3) Subjects with a history of interstitial pneumonia or drug-induced interstitial pneumonia/pneumonitis.

4) Human immunodeficiency virus (HIV) infection.

5) Subjects who are positive for hepatitis B surface antigen (HBsAg) or hepatitis C virus antibody. If negative for HbsAg and positive for the anti-hepatitis B core antibody, a hepatitis B virus-deoxyribonucleic acid (DNA) viral load should not be detected in order to be eligible to participate in the clinical trial.

6) Subjects with chronic liver disease or chronic hepatitis (Child-Pugh class B or C hepatic impairment).

7) Any gastrointestinal disorders which interferes with the absorption of the test drug or makes it impossible to swallow tablets or capsules.

8) Malignant disease, other than that being treated in this clinical trial. Exceptions to this exclusion criteria include the following:
malignancies that were treated curatively and have not recurred within 2 years prior to administration of the test drug.
completely resected basal cell and squamous cell skin cancers.
any malignancy considered to be indolent and that has never required therapy.
completely resected carcinoma in situ of any type.

9) Prior PI3K inhibitor is accepted in the dose escalation part of the study (Phase Ia) only.

10) For subjects with lymphoma:
Systemic antineoplastic therapy (including cytotoxic chemotherapy, α-interferon [INF], and toxin immunoconjugates) or any experimental therapy within 3 weeks or 5 half-lives, whichever is shorter, before administration of a first dose of the test drug.
Therapy with tyrosine kinase inhibitor within 5 half-lives before administration of the first dose of the test drug.
Unconjugated monoclonal antibody therapies<6 weeks before administration of the first dose of the test drug.

11) For subjects receiving a systemic chronic steroid therapy or any immunosuppressive therapy (>10 mg/day prednisone or equivalent). Topical, inhaled, nasal, and ophthalmic steroids are allowed.

12) Use of any live vaccines against infectious diseases within 4 weeks of test drug administration.

13) Use of hematopoietic colony-stimulating growth factor (e.g., granulocyte colony stimulating factor (GCSF), granulocyte-macrophage colony stimulating factor (MCSF)), macrophage colony stimulating factor (MCSF)), thrombopoietin mimetics, or erythroid-stimulating agents≤2 weeks prior to the start date of administration of the test drug. If the administration of an erythrocyte stimulant has been started at least two weeks before a day of first dosing of the test drug and subjects are on a stable dose, the stimulant may be continuously used.

14) Subjects with a history of stroke or having active neurological symptoms (with the exception of chronic conditions which, in the opinion of the neurologist, Investigator, and the Sponsor, would not impact ongoing neurologic assessments while on clinical trial).

15) Active infections requiring a systemic therapy or an antiviral antibiotic therapy.

16) Subjects with active cytomegalovirus (CMV) infection.

17) Subjects receiving moderate or potent CYP3A4 inhibitors or inducers and are unable to withdraw until 2 weeks or 5 times longer than the half-life, whichever is shorter, before the first dose of test drug treatment.

18) Major surgery performed within two weeks before a day of first dosing of the test drug (mediastinoscopy, insertion of a central venous access device, and insertion of a feeding tube are not considered major surgery).

19) Radiotherapy within 2 weeks of first dose of the test drug, except for palliative radiotherapy to a limited-field, such as for the treatment of bone pain or a focally painful tumor mass.

20) Presence of CTCAE≥Grade 2 toxicity (except alopecia, peripheral neuropathy, and ototoxicity, which are excluded if≥CTCAE Grade 3) due to prior cancer therapy.

21) Participation in an interventional, investigational study within 2 weeks or 5 half-lives, whichever is shorter, of the first dosing of the test drug.

22) Any medical condition that would, in the investigator's judgment, prevent the subject's participation in the clinical trial due to safety concerns, compliance with clinical trial procedures, or interpretation of test results.

23) Pregnant or lactating women, where pregnancy is defined as the state of a female after conception and until the termination of gestation, confirmed by a positive human chorionic gonadotropin laboratory test.

### 2. Clinical Trial Design

### A. Test Drug

The test drug is 4-[[(1S)-1-(4,8-dichloro-1-oxo-2-phenyl-3-isoquinolyl)ethyl]amino]-8H-pyrido[2,3-d]pyrimidin-5-one (Table 1).

TABLE 1

| | Test drug |
|---|---|
| IUPAC name | 4-[[(1S)-1-(4,8-dichloro-1-oxo- |
| | 2-phenyl-3-isoquinolyl)ethyl]amino]- |
| | 8H-pyrido[2,3-d]pyrimidin-5-one |
| Dosage formulation | White hard capsules (white on both the |
| | top and the bottom) containing |
| | white to pale yellow powder |
| Manufacturer | Boryung Pharmaceutical Co., Ltd. |

### B. Dosage and Dosing Regimen

A total of 12 subjects enrolled into the clinical trial were orally administered with the test drug once daily for a 28-day cycle (open-label, multicenter) (Table 2).

TABLE 2

| Cohort no. (# of subjects) | Proposed Daily Dose of test drug (QD) |
|---|---|
| Cohort 1 (1) | 50 mg |
| Cohort 2 (1) | 100 mg |
| Cohort 3 (3) | 200 mg |
| Cohort 4 (4) | 325 mg |
| Cohort 5 (3) | 200 mg (Dose expansion) |

In this clinical trial, a starting dose (50 mg QD) of the test drug was determined based on the results of preclinical tests on animals (safety, tolerability and pharmacokinetic data) and toxicity and efficacy obtained from drugs with a mechanism similar to that of PI3K-δ inhibitors in clinical trials.

The dosing regimen used in this clinical trial may be changed if it is judged that more frequent administration, such as BID, is appropriate in consideration of new safety, tolerability, and pharmacokinetic data. In addition, the dosage of this clinical trial may be changed in consideration of a total daily exposure of a new regimen (area under the concentration-time curve, AUC), as long as the dosage does not exceed the established AUC to be safe and tolerable after QD administration (Table 3).

TABLE 3

| | | Increment from Previous |
|---|---|---|
| Dose Level | PK Guided Proposed Daily BID Dose of Test Drug^{a} | Dose |
| 1 | Total daily dose to target approximately ⅔ to ¾ of AUC₍₀₋₂₄₎ | |
| | shown to be safe and tolerated when given QD | |
| 2 | Approximately 0.5 × Dose Level I | 50%/67%/100% |
| | up to an approximately 0.67 × increase is acceptable if the total | |
| | daily dose does not exceed 300 mg day; | |
| | up to an approximately 1 × increase is acceptable if the total daily | |
| | dose does not exceed 200 mg/day | |
| 3 | Approximately 0.33 × Dose Level II | 33%/50%/67% |
| | up to an approximately 0.50 × increase is acceptable if the total | |
| | daily dose does not exceed 350 mg/day; | |
| | up an approximately 0.67 × increase is acceptable if the total | |
| | daily dose does not exceed 300 mg/day; | |
| 4 | Algorithm to follow Dose Level III | 33 to 38% |

Abbreviations: AUC₍₀₋₂₄₎ = area under the concentration-time curve from zero to 24 hours postdose: BID = twice-daily; PK = pharmacokinetics; QD = once-daily.
^{a}It is possible that the SMC may recommend that these levels be modified based on the experience of QD dosing. Preliminary dose escalating algorithm assumes dose-proportional PK as well as entry into the BID algorithm after completion of at least 3 once-daily dose levels.

Precautions for administering the test drug are as follows:

1) Subjects need to take the test drug at least two hours before meals or two hours after meals according to the investigator's instructions.

2) If vomiting occurs during administration, no re-dosing of the subject is allowed before the next scheduled dose.

3) For the QD regimen, if the subject forgets to take his/her dose and can take the dose within 12 hours of the usual time of administration, the dose can be taken. If not, then the dose should be withheld that day and restarted on the next scheduled day. For the BID regimen, if applicable, the dose can only be taken within 6 hours of the usual time of administration.

4) On days when serial blood samples are to be collected for PK profile analyses Cycle 1 Days 1 and 15), subjects are instructed to take the test drug in a fasting state (no food or water after midnight) and refrain from eating for 2 hours following the test drug administration. After this period, food intake is allowed.

5) On the day of PET scans, the subject is required to fast from 6 hours before the PET scan but will be allowed water ad libitum. If a test drug dose is planned on that day the following will apply:
If a scan is scheduled in the morning, the test drug may be taken with water, but the subjects are required not to eat breakfast (usually following the instructions of the investigator).
If the PET scan is scheduled for the afternoon, the subjects may have a light breakfast 4 hours (or more) before the PET scan appointment and take the test drug dose with water 4 hours after the light breakfast. After taking the dose of test drug, the subjects will be required to continue to fast until the PET scan is completed.

### 3. Overall Design

This phase I clinical trial is a multicenter, open-label, and first-in-human (FIH) study consisting of two trial parts (phases Ia and Ib).

The phase Ia part (dose escalation) of this clinical trial is designed to determine safety, tolerability, and maximum tolerated dose (MTD)/starting dose (RP2D) for clinical phase Ib/II of the test drug in subjects with relapsed/refractory B-cell lymphoma, chronic lymphocytic leukemia (CLL)/small lymphocytic leukemia (SLL) and peripheral T-cell lymphoma (PTCL).

The phase Ib part (dose expansion) of this clinical trial is designed to assess a tumor response and safety in specific advanced relapsed/refractory B-cell lymphoma, chronic lymphocytic leukemia (CLL)/small lymphocytic leukemia (SLL) and peripheral T-cell lymphoma (PTCL), at a dose of the test drug identified in phase Ia part. Once the recommended dose for expansion (RDE) has been determined in phase Ia (dose escalation), the phase Ib part (dose escalation) commence.

A planned total trial period for each subject enrolled into the entire clinical trial is approximately 10 months. For subjects without disease progression, the trial period may be extended and the entire clinical trial consists of a screening period, a test drug administration period, and a follow-up period. The details of each period are as follows.

### A. Screening Period

Informed consent was obtained prior to any clinical trial-related procedures or evaluations. After signing the informed consent, all potential subjects were screened (Days −21 to −1).

### B. Test Drug Treatment Period

1) Treatment Period

The test drug treatment period begun at the first dose of the test drug on day 1 of cycle 1, and was consist of consecutive 28-day cycles of the test drug (QD or other regimens determined by the safety monitoring committee) administered orally in a capsule form. Here, one cycle consists of 28 days. Subjects participating in the clinical trial may continue to receive the administration of the test drug until the disease progresses and dose interruptions of the test drug follows the clinical trial guidelines and the investigator's judgment.

2) How to Escalate Dose of Test Drug

A method for escalating a dose during the treatment period (phase Ia part) is as follows. At least of 1 and up to 6 subjects may be enrolled in cohorts 1 and 2. 3 to 6 subjects may be enrolled into subsequent cohorts. The first clinical trial subject in each cohort is treated and observed for 7 days before the test drug is administered to the next 2 clinical trial subjects in that cohort.

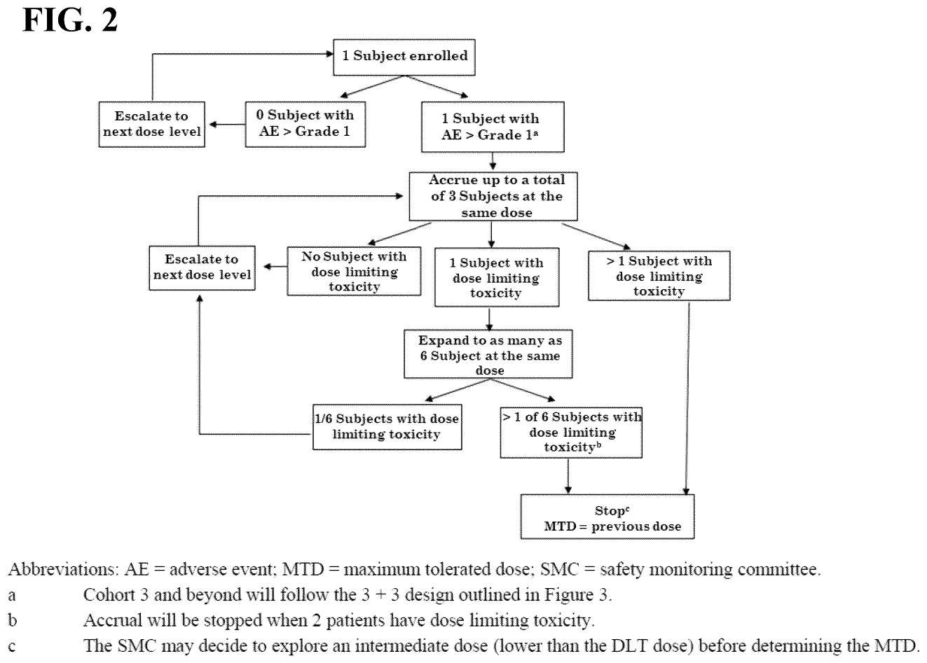

(1) If the first enrolled subject in cohort 1 experiences a Grade 2 or higher adverse event (AE) related to the test drug, additional subjects are enrolled at that cohort and all subsequent cohorts have a planned minimum of 3 subjects. If cohort 1 is successfully completed with only 1 subject, dose escalation will occur, and cohort 2 may start with 1 subject. Then if this first enrolled subject in cohort 2 experiences a Grade 2 or higher AE related to the test drug, additional subjects will be enrolled at that cohort and all subsequent cohorts have a planned minimum of 3 subjects (FIG. 2).

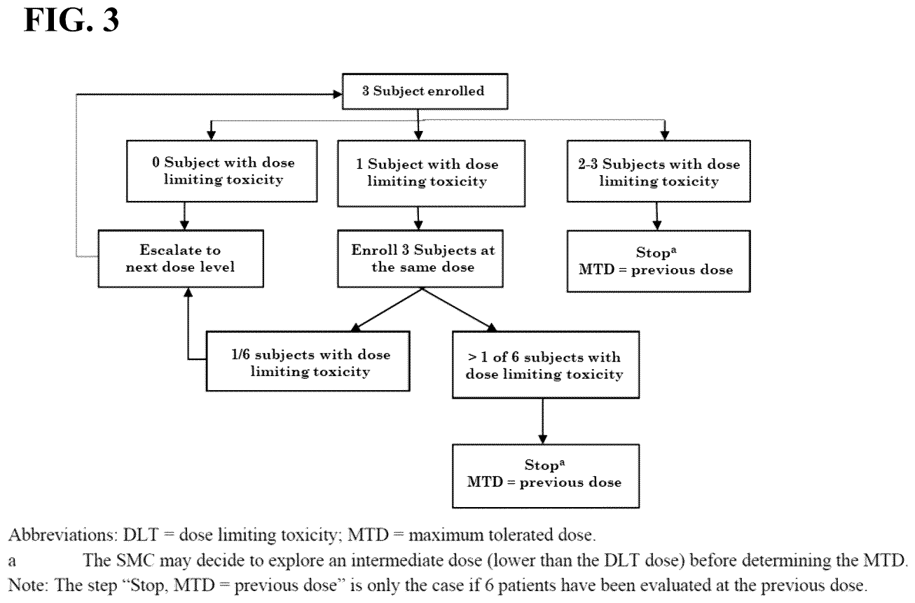

(2) Cohort 3 and all subsequent cohorts will follow the standard 3+3 design. For cohorts enrolling 3 subjects, if there are no dose limiting toxicity (DLT) in the first 3 subjects, dose escalation to the next higher dose level may proceed upon recommendation by the safety monitoring committee (SMC). If 1 of the 3 subjects experiences a DLT, that dose level will be expanded to 6 subjects (3 additional subjects will be enrolled). If 2 subjects at a given dose level experience a DLT, the maximum tolerated dose (MTD) will have been exceeded and further accrual to the cohort will be terminated. The next lower dose level will be expanded to 6 subjects or if 6 subjects have already been enrolled and no more than 1 of the 6 experienced a DLT, then this lower dose level will be considered the MTD (FIG. 3).

(3) A subject experiencing a dose limiting toxicity (DLT) may continue treatment with a reduced dose of the test drug once the DLT is resolved or if the DLT decreases to starting grade or Grade 1 (whichever is greater) prior to initiating the reduced dose. Should a dose-reduced subject experience another DLT, the subject should not receive further doses.

(4) Premature drop-outs are those subjects discontinued in cycle 1 who had <20 of the 28 scheduled days of dosing, as follows:

\textcircled{1} subjects who discontinued due to causes nominally unrelated to adverse events or unrelated to toxicity;

\textcircled{2} subjects who discontinued due to causes other than adverse events or toxicity

(5) Premature drop-outs trigger addition of new subject in any cohort. This decision is to be made after discussion by the clinical trial team, sponsor and investigator and the addition will follow as below.

\textcircled{1} If the subject is in a cohort planned for a single subject (cohorts 1 or 2), the premature drop-out will trigger the single-subject cohort to proceed to standard 3+3 design. Hence, the cohort will be expanded to a planned minimum of 4 subjects. Subsequent cohorts will be planned to have a minimum of 3 subjects following the standard 3+3 design. One DLT in this group will prompt expansion with an additional 3 subjects as in the standard 3+3 description above.

\textcircled{2} If the premature drop-out subject is part of a cohort planned for 3 subjects, the cohort will be expanded to 4 subjects. One DLT in this group will prompt expansion with an additional 3 subjects as in standard 3+3 design.

\textcircled{3} If a subject drops out prematurely and the SMC does not recommend the adding of an additional subject to the cohort, the dropped-out subject will be treated as having experienced a DLT for this dose escalation decision process.

(6) The above dose limiting toxicity (DLT) corresponds to abnormal laboratory value of National Cancer Institute Common Terminology Criteria for Adverse Events (CTCAE) Grade≥3 which the investigator has assessed as unrelated to disease, disease progression, intercurrent illness, or concomitant medications, which occurs within the first cycle of treatment with the test drug during the Phase Ia (dose escalation) part of the clinical trial. The dose limiting toxicity (DLT) in this clinical trial may include the followings:

\textcircled{1} Any grade 5 toxicity.

\textcircled{2} Hematologic dose limiting toxicities.

(i) Any ≥Grade 4 hematologic toxicity except Grade 4 neutropenia lasting≤5 days, lymphopenia, or leukopenia at any grade.

(ii) ≥Grade 3 febrile neutropenia (an ANC<1000/mm3 and a single temperature of >38.3° C. (101° F.) or a sustained temperature of ≥38° C. (100.4° F.) for more than 1 hour).

(iii) Grade 3 neutropenia with infection.

(iv) Grade 3 thrombocytopenia with bleeding.

\textcircled{3} Non-hematologic dose limiting toxicities.

(i) Any ≥Grade 3 toxicity during DLT period, excluding:
Grade 3 diarrhea of <3 days duration following optimal therapy.
Grade 3 nausea and vomiting of <3 days duration with optimal therapy.
Grade 3 fatigue<7 days duration following initiation of adequate supportive care.
Asymptomatic Grade 3 liver function test (alanine aminotransferase [ALT], aspartate aminotransferase [AST], or gamma-glutamyl transferase [GGT]) increase that resolves to ≤Grade 1 in less than 7 days after medical management.
Any other laboratory values out of normal range that are unlikely related to study treatment, do not have any clinical correlation, and resolve to ≤Grade 1 within 7 days with adequate medical management.

(ii) Grade 3 or higher skin reactions including dermatitis exfoliative, rash erythematous, rash generalized, rash macular, rash papular, exfoliative rash and skin disorders.

(iii) Grade 2 pneumonitis which does not resolve within seven days after medical management.

\textcircled{4} Any clinically significant or persistent toxicities that may also be considered DLTs following review by the safety monitoring committee (SMC).

### C. Follow-Up Period

The follow-up period consists of a safety follow-up visit at 30±2 days after administration of the last dose of the test drug, a subsequent follow-up visit (if necessary), and an end-of-study (EOS) visit. Subjects who discontinue treatment should be followed up until resolution of toxicity or until confirmed disease progression.

### 4. Assessment Method

### A. Test Procedure and Assessment

The trial assessment, procedure, and the schedule of activities are as summarized in table 4, and at scheduled time points, where multiple assessments are simultaneously scheduled, the order of assessments is as follows: electrocardiogram (ECG), vital signs, then blood sampling (PK sampling followed by PD sampling and safety laboratory sampling).

TABLE 4

| | Visit name | | | | | | | | | | | | | | |
|---|---|---|---|---|---|---|---|---|---|---|---|---|---|---|---|
| | | Treatment phase | | | | | | | | | | | | | |
| | | Day of cycle | | | | | | | | | | | | | |
| | | | | | | | | | Cycle | | After 6 | Follow-up | | | |
| | Screening | Cycle 1^{c} | | | | | Cycle 2 | | 3 to 6 | | cycles | | 30-day | | |
| | Screening^{b} | 1 | 2 | 8 | 15/16 | 22 | 1 | 15 | 1 | 15 | 1 | EOT^{d} | safety | FU^{e} | EOS^{f} |
| | Windows (days) | | | | | | | | | | | | | | |
| | −21 to −1 | ±2 | ±2 | ±2 | ±2 | ±2 | ±2 | ±2 | ±2 | ±2 | ±2 | +7^{d} | ±2 | ±2 | ±2 |
| Obtain informed | X | | | | | | | | | | | | | | |
| consent (includes | | | | | | | | | | | | | | | |
| biomarker and | | | | | | | | | | | | | | | |
| study-related | | | | | | | | | | | | | | | |
| biopsy consent) | | | | | | | | | | | | | | | |
| Subject number | X | | | | | | | | | | | | | | |
| alignment | | | | | | | | | | | | | | | |
| Subject History | | | | | | | | | | | | | | | |
| Demography | X | | | | | | | | | | | | | | |
| Inclusion/exclusion | X | | | | | | | | | | | | | | |
| criteria | | | | | | | | | | | | | | | |
| Medical history | X | | | | | | | | | | | | | | |
| Diagnosis and | X | | | | | | | | | | | | | | |
| extent of cancer | | | | | | | | | | | | | | | |
| Prior antineoplastic | X | | | | | | | | | | | | | | |
| therapy | | | | | | | | | | | | | | | |
| Prior and concomitant | Continuous | | | | | | | | | | | | | | |
| medications | | | | | | | | | | | | | | | |
| Physical examination | X^{g} | X^{g} | | | X^{h} | | X^{h} | X^{h} | X^{h} | X^{h} | X^{h} | X^{h} | X^{h} | X^{h} | X^{h} |
| Heightⁱ | X | | | | | | | | | | | | | | |
| Weightⁱ | X | X | | | | | X | | X | | X | X | X | X | X |
| Vital signs | X | X^{j} | X^{j} | X^{j} | X^{j} | X^{j} | X^{j} | X | X^{l} | X | X | X | X | X | X |
| ECOG performance status | X | X | | | X | | X | X | X | X | X | X | X | X | X |
| Bone marrow biopsy^{k} | X | | | | | | | | | | | | | | |
| Laboratory assessments [symbol] | | | | | | | | | | | | | | | |
| Hematology | X | X | | X | X | X | X | X | X | X | X | X | X | X | X |
| Chemistry including | X | X | | X | X | X | X | X | X | X | X | X | X | X | X |
| fasting glucose | | | | | | | | | | | | | | | |
| Coagulation | X | X | | | X | | X | | X | | X | X | X | X | X |
| Thyroid function test | X | X | | | X | | X | | X | | X^{l} | X | | X | |
| Urine analysis | X | X | | | X | | X | | X | | X | X | X | X | |
| HbA1c | X | X | | | | | | | | | X^{m} | X | | X | |
| Virology (HIV; HBV, | X | | | | | | | | | | | | | | |
| and HCV) | | | | | | | | | | | | | | | |
| CMV testing [symbol] | X | During treatment: CMV surveillance will be performed as per local practice. | | | | | | | | | | | | | |
| Pregnancy test (females)^{o} | X | | | | | | X | | X | | X | X | X | X | X |
| Follicle-stimulating | X | | | | | | | | | | | | | | |
| hormone and estradiol | | | | | | | | | | | | | | | |
| (females)^{p} | | | | | | | | | | | | | | | |
| Paraprotein blood sample^{q} | X | During treatment: every 2 cycles starting on Cycle 3 Day 1 until 12 cycles | | | | | | | | | | | | X | X |
| | | are reached, then every 4 cycles until disease progression. The sample will | | | | | | | | | | | | | |
| | | be collected prior to the regular visit at the same timepoint of tumor | | | | | | | | | | | | | |
| | | disease evaluation. Unscheduled tests may be performed at the | | | | | | | | | | | | | |
| | | discretion of the Investigator. | | | | | | | | | | | | | |
| Imaging^{r} | | | | | | | | | | | | | | | |
| Tumor disease evaluation^{s} | X | Every 2 cycles during first 12 cycles, then every 4 cycles thereafter. | | | | | | | | | | | | X | X^{t} |
| | | The PET scan should be performed a minimum of 3 to a maximum of 10 | | | | | | | | | | | | | |
| | | days prior to blood sampling to avoid radioactive contamination of the | | | | | | | | | | | | | |
| | | blood sample. The tumor disease evaluation will be performed before start | | | | | | | | | | | | | |
| | | of each cycle 3, 5, 7, 9, 11 and 13, then cycles 17, 21, 25 and so on. | | | | | | | | | | | | | |
| | | Unscheduled test may be performed at the discretion of the Investigator. | | | | | | | | | | | | | |
| Electrocardiogram | X | X^{j} [symbol] | X^{j} | X^{j} | X^{j} | X^{j} | X^{j} [symbol] | | X^{j} [symbol] | | X^{o} | X | X | X | X |
| MUGA or ECHO | X | | | | | | | | | | | | | | |
| Tolerability | | | | | | | | | | | | | | | |
| Adverse Events | X^{v} | Continuous | | | | | | | | | | | | | |
| Pharmacodynamics | | | | | | | | | | | | | | | |
| Collection of archival | X | | | | | | | | | | | | | | |
| or fresh tumor sample | | | | | | | | | | | | | | | |
| at baseline^{w} | | | | | | | | | | | | | | | |
| Collection of new tumor | | | | | | | X | | | | | | | | |
| sample^{w} | | | | | | | | | | | | | | | |
| Blood sample for immune | X | X | | | X | | X | | X | | | X | | X | |
| response (whole blood | | | | | | | | | | | | | | | |
| PD biomarkers)^{x} | | | | | | | | | | | | | | | |
| Blood sample for PD | X | X | | | X | | X | | X | | | X | | X | |
| markers (cytokines and | | | | | | | | | | | | | | | |
| chemokines)^{x} | | | | | | | | | | | | | | | |
| Serum β2-microglobulin | X | X | | | | | X | | X | | X | X | X | X | X |
| Serum immunogloblins: | X | During treatment: every 2 cycles starting on Cycle 3 Day 1 until 12 | | | | | | | | | | | | X | X |
| IgM, IgG, and IgA [symbol] | | cycles are reached, then every 4 cycles until disease progression. | | | | | | | | | | | | | |
| | | The sample will be collected prior to the regular visit at the same | | | | | | | | | | | | | |
| | | timepoint of tumor disease evaluation. Unscheduled tests may be | | | | | | | | | | | | | |
| | | performed at the discretion of the Investigator. | | | | | | | | | | | | | |
| Eligibility check | | | | | | | | | | | | | | | |
| Allocation of study | | X | | | | | X | | X | | X | | | | |
| treatment (using IRS | | | | | | | | | | | | | | | |
| system) | | | | | | | | | | | | | | | |
| Test drug treatment | | | | Oral single daily dose administration (dose regimen may | | | | | | | | | | | |
| administration | | | | be adjusted depending on emerging safety, tolerability, | | | | | | | | | | | |
| | | | | PK, and PD data) | | | | | | | | | | | |
| PJP prophylaxis [symbol] | | | | Prophylaxis will start on Day 1 of Cycle 1 and | | | | | | | | | | | |
| | | | | will continue throughout the treatment period. | | | | | | | | | | | |
| Compliance check^{z} | | | | | X | | X | | X | | X | X | | | |
| Blood samples for PKY [symbol] | | X^{aa} | X^{aa} | X | X^{aa} | X | X | | X^{bb} | | X^{bb} | X | | | |
| Urine samples for PKN [symbol] | | X^{aa} | | | X^{aa} | | | | | | | | | | |
| Antineoplastic therapy | | | | | | | | | | | | | X^{cc} | | X^{cc} |
| since discontinuation of | | | | | | | | | | | | | | | |
| study treatment | | | | | | | | | | | | | | | |

Abbreviations: CLL = Chronic lymphocytic leukemia; CMV = Cytomegalovirus; CR = complete response; CSR = clinical study report; CT = computed tomography; D = clinical database; ECG = electrocardiogram; ECHO = echocardiogram; ECOG = Eastern Cooperative Oncology Group; EOS = End of Study; EOT = End of Treatment; FU = Follow-up; HbA1c = glycated hemoglobin; HBV = hepatitis B virus; HCV = hepatitis C virus HIV = human immunodeficiency virus; IRS = interactive response system; MRI = magnetic resonance imaging; MUGA = multiple gated acquisition scan; PCR = polymerase chain reaction; PD = pharmacodynamic; PET = positron emission tomography; PJP = Pneumocystis [symbol] pneumonia; PK = pharmacokinetic; S = source documents.
^{b}Screening/Baseline evaluations must be performed ≤21 days before Cycle 1 Day 1 (1 cycle = 28 days), except for the pregnancy test in women of child bearing potential winch will be performed within 72 hours before first dose of test drug treatment. Laboratory assessments performed as part of the screening/baseline evaluations and within 3 days prior to the first dose of study treatment, are not required to be repeated on Cycle 1 Day 1. Laboratory and radiological assessments performed as part of standard of care prior to signing informed consent may be used if performed within the screening/baseline time window.
^{c}Subjects may be required to be hospitalized in Cycle 1 Day 1 to 2 and Day 15 to 16. All assessments on Day 1 of Cycle 1 will be performed predose. Cycle 1 Day 2 and Cycle 1 Day 16 is applicable for Phase Ia and first 8 to 10 subjects in Phase Ib.
^{d}The EOT Visit will occur within 7 days after the last dose of study treatment or on the day visit of the decision of discontinue study treatment. After primary CSR cut-off is reached the following assessment should no longer be done at EOT or subsequent follow-up timepoints: ECG and PK sample.
^{e}If the subject discontinues treatment but continues to the study, a follow-up visit should be performed at every 56 days from the previous response assessment until disease progression is continued. The 30-day Safety Visit and Follow-up Visit can be combined in necessary.
^{f}EOS Visit will be performed when the subject is discontinued from the study (disease progression is confirmed or consent is withdrawn by the subject). If new alternative treatment is required, the subject should be discontinued from the study. If the subject is discontinued the study at the same time of treatment discontinuation, only the EOT Visit will be performed.
^{g}A complete physical examination will be performed and will include the examination of general appearance, skin, neck (including thyroid), eyes, ears, nose, throat, lungs, heart, abdomen, back, lymph nodes, extremities, and neurological. If medicated based on medical history and/or symptoms, rectal, external genitalia, breast, and pelvic examinations will be performed.
^{h}A short/focused physical examination consisting of the examination of general appearance and body [symbol] as directed by symptoms will be performed, unless the Investigator considers a complete physical examination necessary.
ⁱBody mass index (weight [kg]/height² [m × m]) will be calculated for screening measurements.
^{j}Vital sign and 12-lead ECG assessment times for Cycle 1, 2 and 3 are listed in detail in Table 9. Vital sign should be collected before blood sampling. (Note: Vital sign should be performed on Day 15 of Cycle 2 and 3 as per Table 3.)
^{k}Bone marrow biopsies will be performed at baseline for CLL patients (subjects) (in the event a biopsy is not possible, an aspirate will be acceptable). For all other subjects this is not mandatory but is recommended. Lymphoma patients (subjects) who have positive baseline bone marrow involvement will need a repeat bone marrow biopsy in case of CR to confirm CR.
^{l}If clinically needed.
^{m}Every 6 cycles starting on Cycle 7 Day 1.
ⁿCMV testing (serology and/or antigen) will be performed at Screening per local standard practice. Subjects with active CMV infection will not be enrolled. CMV surveillance with CMV antigen and/or PCR testing will be performed as per local practice. If CMV testing is unable to be performed locally, testing will be performed by a central laboratory.
^{o}A serum pregnancy test will be performed for women of child bearing potential at screening (within 3 days before the first dose), on Day 1 of each cycle starting with Cycle 2, EOT, and the Follow-up Visit.
^{p}As needed in women of non-childbearing potential only (postmenopausal women aged over 45 years with at least 1 year of [symbol] )
^{q}Subjects with Waldenstrom macroglobulinemia only, serum M-protein by serum protein electrophoresis with immunofixation at baseline and every disease assessment. In addition, serum immunoglobulins will only be measured in subjects with Waldenstrom macroglobulinemia. The Investigator should confirm the results are available before the scheduled visit same timepoint as per imaging scan.
^{r}Any imaging assessments (PET-CT, diagnostic quality contrast CT, or MRI) already completed during the regular work-up of the subject within 21 days prior to start of treatment, including before signing the main study informed consent form, can be considered as the baseline image for this study.
^{s}Imaging scans (PET-CT, contrast CT, or MRI, consistent mode should be used throughout the study) will be performed at baseline for all subjects. Position emission tomography-CT scans should be performed a minimum of 3 to a maximum of 10 day before blood sampling to avoid radioactive contamination of the blood sample. The Investigator should confirm the imaging scan results are available before the scheduled visit.
PET-CT scan will be required as standard baseline procedure for lymphomas only using Lugano criteria. For PET-avid disease a repeat PET-CT scan will be performed to verify CR
For other hematologic malignancies, PET scan is not required. The contrast CT will be the standard imaging scans for these hematologic malignancies.
Bone marrow aspirates biopsy may be performed at baseline if needed. Lymphoma patients (subjects) who have positive baseline bone marrow involvement will need a repeat bone marrow aspirate in case of CR to confirm CR.
For Waldenstrom macroglobulinemia, repeat bone marrow aspirate will be performed in case of complete remission to confirm complete remission.
For CLL, repeat bone marrow aspiration will be performed in case of CR or Minimal Residual Disease.
Serum M-protein by serum protein electrophoresis with immunofixation for Waldenstrom macroglobulinemia will be performed at baseline and every disease assessment.
^{t}Tumor evaluation does not need to be repeated if the Investigator has determined that the subject has disease progression.
^{u}Day 1 pf each cycle. The measurement of 12-lead ECG will be performed before vital signs and before any blood sample collections.
^{v}Medical occurrences that begin before the start of study treatment but after obtaining informed consent will be reconted on the Medical History/Current Medical Conditions section of the CRF. Events related to study procedures after obtaining informed consent will be reported as AEs or SAEs, as appropriate.
^{w}Paired tumor biopsies will be obtained from 1 to 2 subjects with non-liquid tumors per cohort in Phase Ia (dose escalation) and from approximately 14 subjects in Phase Ib (dose expansion). Sampling handling instructions will be provided. For lymphoma patients (subjects), formalin-fixed, paraffin-embedded tissue block or at least 15 unstained slides for submission.
^{x}Blood and urine sample collections for PD and PK analyses and acceptable time windows are listed in Table 5.
^{y}Subjects must receive trimethoprim-sulfamethoxazole or other established prophylaxis as per standard practice for PJP during treatment period.
^{z}Compliance will be assessed on Day 1 of each cycle with [symbol] study medication and subject diary card. Diary cards will be completed for Cycle 1 only. Training will be provided by study center staff on how to complete the diary card. The diary cards will be provided to the subject on Day 8 of Cycle 1 and retrieved on Day 15/16 of Cycle 1 (or Day 22 of Cycle 1/Day 1 of Cycle 2 if PK sampling is delayed).
^{aa}Serial blood sample collection for PK and PD analysis to be performed on Day 1/2 and Day 15/16 and urine sample collection on Day 15/16 over the entire dosing interval. To accommodate the suject's schedule, the Day 15/16 PD sample collection, plasma and urine PK collections may be delayed to either the Day 22 visit or Day 1 of Cycle 3, 6, 9, and 12 only.
^{bb}Day 1 of Cycle 3, 6, 9, and 12 only.
^{cc}Alternative anticancer therapy information if available may be collected in EOS when the subject is discontinued from the study. [symbol]
indicates data missing or illegible when filed

### B. Primary Endpoints . . . Assessment of Safety and Tolerability

1) Assessed by adverse events, vital signs, electrocardiogram (ECG), clinical laboratory tests, physical examination, ECOG performance status up to the last Safety Follow-up Visit, and baseline medical conditions.

2) Tolerability assessment includes review of dose interruptions, reductions, and doses administered up to the last Safety Follow-up Visit.

3) Determination of the maximum tolerated dose (MTD) and the starting dose (RP2D) for clinical phase Ib/II are based on dose limiting toxicity (DLT) during Cycle 1.

### C. Secondary Endpoints . . . Plasma and Urine PK Characterization and Preliminary Antitumor Activity Assessment

1) Plasma concentration-time profile of the test drug (days 1 and 15), urine concentration data (day 15), and derived single dose plasma PK and multiple dose plasma and urine PK parameters (if applicable).
Blood samples are collected according to a predetermined schedule to measure a plasma concentration of the test drug (Table 5). In phase Ia clinical trials, entire urine needs to be collected over each dosing interval according to a pre-determined schedule, starting after dose administration (Table 5).

TABLE 5

| | | | Phase Ia and the first 8 | | | | |
|---|---|---|---|---|---|---|---|
| | | | to 10 subjects of Phase Ib | | | | |
| | | | Blood samples | Pooled urine | | Remaining Phase Ib subjects | |
| | | Scheduled time point (PK blood | for test drug and | samples for | PD | Test drug and | PD |
| Cycle | Day | sample collection windows)^{a} | metabolites^{b} | test drug^{c} | sample | metabolites^{b} | sample |
| 1 | 1 | Predose^{d} | x | Predose | x | x | x |
| | | | | void | | | |
| 1 | 1 | 0.5 hours postdose (±5 minutes) | x | | | | |
| 1 | 1 | 1 hour postdose (±5 minutes) | x | | | | |
| 1 | 1 | 1.5 hour postdose (±5 minutes) | x | | | | |
| 1 | 1 | 2 hours postdose (±0.25 hours) | x | | x | 2 ± 1 | x |
| | | | | | | hour | |
| 1 | 1 | 3 hours postdose (±0.25 hours) | x | | | | |
| 1 | 1 | 4 hours postdose (±0.5 hours) | x | | | | |
| 1 | 1 | 6 hours postdose (±0.5 hours) | x | | x | 6 ± 1 | x |
| | | | | | | hour | |
| 1 | 1 | 9 hours postdose (±0.5 hours) | x | | | | |
| 1 | 1 | 12 hours postdose (±0.5 hours)^{e} | x | | x | | |
| 1 | 2 | 24 hours postdose (±1 hour)^{f} | x | | x | | |
| 1 | 8 | Predose^{d} | x | | | x | |
| 1 | 15 | Predose^{d} | x | Predose | x | x | x |
| | | | | void | | | |
| 1 | 15 | 0.5 hours postdose (±5 minutes) | x | 0 to 6 | | | |
| | | | | hours | | | |
| 1 | 15 | 1 hour postdose (±5 minutes) | x | | | | |
| 1 | 15 | 1.5 hour postdose (±5 minutes) | x | | | | |
| 1 | 15 | 2 hours postdose (±0.25 hours) | x | | x | 2 to 3 | x |
| | | | | | | hours | |
| 1 | 15 | 3 hours postdose (±0.25 hours) | x | | | | |
| 1 | 15 | 4 hours postdose (±0.5 heims) | x | | | | |
| 1 | 15 | 6 hours postdose (±0.5 hours) | x | | x | | |
| 1 | 15 | 9 hours postdose (±0.5 hours) | x | 6 to 12 | | | |
| | | | | hours | | | |
| 1 | 15 | 12 hours postdose (±0.5 hours)^{e} | x | | x | | |
| 1 | 16 | 24 hours postdose (±1 hour)^{f} | x | 12 to 24 | x | | |
| | | | | hours | | | |
| 1 | 22 | Predose^{d} | x | | x | x | x |
| 2 | 1 | Predose^{d} | x | | x | | |
| 2 | 1 | 2 to 4 hours postdose | x | | x | x | x |
| 3 | 1 | Predose^{d} | x | | x | x | x |
| 3 | 1 | 1 to 5 hours postdose | x | | x | x | x |
| 6, 9, and 12 | 1 | Day 1. Anytime during visit at | x | | | x | |
| | | the time blood is collected for | | | | | |
| | | laboratory assessment. | | | | | |
| EOT | | Anytime during visit | x | | x | x | x |

| | | | | | | |
|---|---|---|---|---|---|---|
| Total samples | Through Cycle 1 | 23 | 3 | 11 | 7 | 6 |
| planned | Through Cycle 2 | 25 | | 13 | 9 | 7 |

Abbreviations: AE = adverse event; BID = twice-daily; ECG = electrocardiogram; EOT = End of Treatment; PD = pharmacodynamics; PK = pharmacokinetics; QD = once-daily.
Note: To accommodate the patient's schedule, the Day 15/16 serial PK collections may be delayed to either the Day 22 visit or the Cycle 2, Day 1 visit. A PK sample will also be collected in the event of a clinically significant AE.
^{a}The scheduled times for the PK sample collections may be modified if warranted by the emerging PR results from prior cohorts. If the schedule is modified, the number of PK samples planned over the entire treatment period (excluding unscheduled samples) will not be increased but may be decreased.
^{b}Unused plasma samples may be subjected to further analysis for metabolite profiling of test drug in human plasma.
^{c}In Phase Ia only, subjects will void prior to dosing on Day 1 and on Day 15, these samples will be collected but not analyzed unless unexpected urine results are obtained. Beginning on Day 15, pooled urine will be collected for the following intervals: 0 to 6 hours (beginning immediately after test drug administration) and 6 to 12 hours. For Subjects remaining in the clinic overnight on Day 15, pooled urine samples will be collected over the 12 to 24-hour interval during the in-clinic stay. Subjects who do NOT remain in the clinic overnight on Day 15 will be asked to void and record the time of void just prior to returning to the clinic on Day 16. Upon their return to the clinic for the Day 16 visit at approximately 24-hours postdose, subjects will be asked to void, and the full urine void will be collected. Note: the urine collection from 12 to 24-hours or at 24 hours will only apply for QD dosing.
^{d}Predose samples must be collected within 60 minutes of the first dose on Cycle 1 and within 30 minutes prior to all subsequently scheduled doses.
^{e}12-hour sample must be collected prior to the next dose, if test drug is dosed BID.
^{f}24-hour sample must be taken prior to the Day 2 of Day test drug dose (QD regimen only).

Samples for the determination of the test drug in plasma and urine are analyzed using appropriate validated bioanalytical methods.
Pharmacokinetic parameters are derived using noncompartmental methods with Phoenix® WinNonlin® Version 8.0 or higher (Certara, L.P. Princeton, New Jersey, USA) and/or SAS® Version 9.2 or higher (SAS Institute, Inc., Cary, North Carolina, USA). Actual elapsed time from dosing will be used for the final plasma PK parameter calculations. A minimum of 3 quantifiable postdose concentrations will be required for all calculations.
The PK parameters to be determined for plasma concentration of the test drug on day 1 of cycle 1 and day 15 of cycle 1, if calculable, include but not be limited to the following parameters presented table 6.

TABLE 6

| Plasma | |
|---|---|
| pharmacokinetic | |
| parameter | Definition |
| Cₘₐₓ | Maximum concentration obtained directly from the observed concentration versus time |
| | data. |
| tₘₐₓ | Time to C_{max.} |
| Cₘᵢₙ | Minimum concentration obtained directly from the observed concentration versus time |
| | data (Day 15 only). |
| C_{avg} | Average concentration over a dosing interval computed as (AUC_{(0-tau), D 15}/tau) (Day 15 |
| | only) |
| AUC_{(0-inf)} | Area under the plasma concentration-time curve from time zero extrapolated to infinity. |
| | calculated by linear up log down trapezoidal summation (Day 1 only). |
| AUC₍₀₋ₗₐₛₜ₎ | Area under the plasma concentration-time curve from time zero to the time of the last |
| | quantifiable concentration, calculated by linear up/log down trapezoidal summation. |
| AUC₍₀₋ₜₐᵤ₎ | Area under the plasma concentration-time curve from time zero during a dosing interval |
| | (24 or 12 hours), calculated by linear up log down trapezoidal summation. |
| CL/F | Apparent oral plasma clearance (parent only). |
| V₂/F | Apparent volume of distribution (parent only). |
| t_{1/2} | Terminal half-life: a minimum of 3 points will be used for estimation. |
| λ_{z} | Terminal rate constant. |
| RCₘₐₓ | Accumulation ratio for Cₘₐₓ computed as (C_{max, D 15}/C_{max, D 1}). |
| R_{AUC(0-tau)} | Accumulation ratio for AUC₍₀₋ₜₐᵤ₎ computed as (AUC_{(0-tau), D 15}/AUC_{(0-tau), D 1}), |
| LI | Time Imearity index computed as (AUC_{(0-tau), D 15}/AUC_{(0-inf), D 1}). |

The PK parameters to be determined for urine test drug concentration on cycle 1 day 15, if calculable, include but not be limited to the following parameters presented in Table 7.

TABLE 7

| Urine | |
|---|---|
| Pharmacokinetic | |
| parameter | Definition |
| Ae₍ₜ₁₋ₜ₂₎ | Amount of unchanged drug excreted in urine during each collection interval from time |
| | 1 (t1) to time 2 (t2), calculated as [urine concentration × urine volume]. |
| Ae₍₀₋₁₂₎ | Cumulative amount of unchanged drug excreted in urine from zero (predose) to 12 hours, |
| | calculated as the summation of the amounts excreted in the scheduled collection intervals |
| | through 12 hours. |
| Ae₍₀₋₂₄₎ | Cumulative amount of unchanged drug excreted in urine from zero (predose) to 24 hours, |
| | calculated as the summation of the amounts excreted in the scheduled collection intervals |
| | through 24 hours. (Only applicable for QD dosing). This amount may be estimated by the |
| | excretion rate method for subjects with partial outpatient urine collection on Day 16. |
| fe₍₀₋₁₂₎ | Fraction of dose excreted unchanged in urine from zero (predose) to 12 hours (8%), |
| | calculated as Ae₍₀₋₁₂₎ divided by dose × 100. |
| fe₍₀₋₂₄₎ | Fraction of dose excreted unchanged in urine from zero (predose) to 24 hours (6), |
| | calculated as Ae₍₀₋₂₄₎ divided by dose × 100. |
| CL_{R} | Renal clearance calculated as Ae₍₀₋₂₄₎/AUC₍₀₋ₜₐᵤ₎ with tau = 24 hours for QD dosing of |
| | Ae₍₀₋₁₂₎/AUC₍₀₋ₜₐᵤ₎ with tau = 12 hours for BID dosing. [Note: alternately CL_{R} for QD |
| | dosing in patients with outpatient urine collection may be calculated as Ae₍₀₋₁₂₎/AUC₍₀₋₁₂₎] |

### D. Exploratory Endpoints . . . Pharmacodynamic (PD) Characterization and Biomarker Investigation

1) Absolute and change from baseline in systemic cytokines and chemokines; phenotypic characterization of activated immune cells; changes from baseline in tumor biopsy assessment; and other pharmacodynamic (PD) biomarkers test drug exposure.
Exploratory pharmacodynamic (PD) and biomarker (immunogenicity) data may include analysis of the following:

(i) Systemic cytokines and chemokines (serum): CCL3, CCL4, INF-γ, interleukin (IL)-6, IL-10, tumor necrosis factor (TNF)-α.

(ii) Whole blood pharmacodynamic (PD) biomarkers:

\textcircled{1} Regulatory T cells-programmed cell death protein 1 (PD 1), cytotoxic T cell lymphocyte-associated protein (CTLA)-4, lymphocyte activation gene (LAG)-3.

\textcircled{2} Cluster of differentiation (CD)8-PD 1, CTLA-4, LAG-3, T cell immunoglobulin (Ig) and mucin domain containing-3, T-cell immunoreceptor with Ig and immunoreceptor tyrosine-based inhibition motif domains, Ki67.

\textcircled{3} Myeloid-derived suppressor cells.

(iii) Tumor biopsy: C-MYC (IHC, FISH), rH2AX, PAKT (T308, S473)
Paired tumor biopsy is obtained from 1 to 2 subject(s) with non-liquid tumors per cohort in Phase Ia (dose escalation) and in Phase Ib (dose expansion) according to the planned schedule of activities to explore PD biomarkers. Similar information may be obtained from the standard tumor assessment for liquid tumors. For lymphoma subjects, a formalin-fixed, paraffin-embedded (FFPE) tissue block or at least 15 unstained slides for submission. The investigations that are chosen will depend on the availability of sufficient and suitable biological specimens and will be defined in the laboratory manual.
Exploratory biomarker analysis may be conducted on any remaining tumor and/or blood samples (including PK samples). In this trial, the search for other potentially relevant biomarkers to investigate the effect of the test drug as well as to determine how changes in biomarkers may relate to test drug exposure and clinical outcomes. These additional investigations are dependent upon dependent upon clinical outcome, reagent, and sample availability.

2) Profiling of Test Drug Metabolites

### E. Safety Assessments

1) Safety Assessments Items

Planned time points for all safety assessments are provided in table 4, and the methods are as follows.

(1) Demographic and Medical History

Demographic information and relevant medical and surgical history are recorded in an electronic case report form (eCRF).

(2) Physical Examinations

At screening and cycle 1 day 1, prior to administration of the test drug, complete physical examinations are performed, and are include the examination of general appearance, skin, neck (including thyroid), eyes, ears, nose, throat, lungs, heart, abdomen, back, lymph nodes, extremities, and neurological. If indicated based on medical history and/or symptoms, rectal, external genitalia, breast, and pelvic examinations are performed. At subsequent assessments short physical examinations consisting of the examination of general appearance and body sites as directed by symptoms are performed.

(3) Vital Signs

Vital signs (body temperature, pulse rate, respiratory rate, and blood pressure) are measured in the supine or sitting position during the trial and should be preceded by at least 5 minutes of rest in the supine or sitting position. The position is documented in the eCRF. Vital signs are collected prior to any simultaneously scheduled blood collections (e.g., PK or clinical laboratory samples). Additional vital signs measurements may be performed at any time during the trial at the discretion of the investigator, if medically indicated.

(4) Height and Weight

Height (only at screening) and body weight are measured according to the study center's standard procedures. the body mass index (BMI: weight [kg]/height² [m×m]) is calculated for screening measurements.

(5) Performance Status

Eastern Cooperative Oncology Group performance status (ECOG PS) is assessed as below (Table 8).

TABLE 8

| Grade | ECOG Status |
|---|---|
| 0 | Fully active, able to carry on all pre-disease |
| | performance without restriction |
| 1 | Restricted in physically strenuous activity but ambulatory |
| | and able to carry out work of a light or |
| | sedentary nature (eg, light house work or office work) |
| 2 | Ambulatory and capable of all selfcare but unable to |
| | carry out any work activities. Up and about |
| | more than 50% of waking hours |
| 3 | Capable of only limited selfcare, confined to bed or |
| | chair more than 50% of waking hours |
| 4 | Completely disabled. Cannot carry on any selfcare. |
| | Totally confined to bed or chair |

(6) Electrocardiogram

Duplicate 12—lead electrocardiogram is obtained at times listed in the Schedule of Activities (Table 4) and the times listed in table 9, respectively, after resting for at least 5 minutes using an ECG machine that automatically calculates the heart rate and measures PR, QRS, QT, and QTc intervals. And the following parameters are recorded: Heart rate, PR interval, QRS duration, QT and QTcF.

At each time point, 2 individual ECG tracings should be obtained as closely as possible in succession, but no more than 2 minutes apart. The duplicates should be completed in less than 5 minutes. After the primary CSR cut-off date is reached, it is not necessary to measure electrocardiogram (ECG) with the EOT assessments. Additional ECGs may be conducted for safety reasons at the discretion of the investigator.

(7) Cardiac Imaging

The left ventricular heart function is evaluated by ECHO or MUGA scan at screening/baseline. Additional cardiac imaging may be performed if indicated by clinical signs or symptoms. The same imaging modality should be used for the additional imaging.

(8) Clinical Safety Laboratory Assessments

A list of clinical laboratory tests to be performed is presented below (Table 10). See table 4 for inspection timing and frequency.

TABLE 9

| | | | Phase Ia and the first 8 | | Remaining Phase | |
|---|---|---|---|---|---|---|
| | | | to 10 subjects of Phase Ib | | Ib subjects | |
| | | | | Vital | | Vital |
| Cycle | Day | Scheduled time point | ECG^{a} | signs^{a} | ECG^{a} | signs^{a} |
| 1 | 1 | Predose (within 60 minutes of dose | x | x | x | x |
| | | administration) | | | | |
| 1 | 1 | 0.5 hour postdose | x | x | x | x |
| 1 | 1 | 1 hour postdose | x | x | | |
| 1 | 1 | 2 hours postdose | x | x | x | x |
| 1 | 1 | 4 hours postdose | x | x | | |
| 1 | 1 | 6 hour Phase Ia 5-7 hours prior to | x | x | 6 ± 1 | 6 ± 1 |
| | | the scheduled PK sample in Phase Ib | | | hour | hour |
| 1 | 1 | 9 hours postdose | x | x | | |
| 1 | 1 | 12 hours postdose | x | x | x | x |
| 1 | 2 | 24 hours postdose sample must be taken | x | x | | |
| | | prior to the next dose (QD only) | | | | |
| 1 | 8 | Predose (within 30 minutes of dose | x | x | x | x |
| | | administration) | | | | |
| 1 | 15 | Predose (trough; within 30 minutes | x | x | x | x |
| | | of dose administration) | | | | |
| 1 | 15 | 1 hour postdose | x | x | | |
| 1 | 15 | 2 hours postdose | x | x | 2 to 3 | 2 to 3 |
| | | | | | hours^{b} | hours^{b} |
| 1 | 15 | 4 hours postdose | x | x | | |
| 1 | 15 | 9 hours postdose | x | x | | |
| 1 | 16 | 24 hours postdose sample must be taken | x | x | | |
| | | prior to the next dose (QD only) | | | | |
| 1 | 22 | Predose | x | x | x | x |
| 2 | 1 | Predose | x | x | x | x |
| 3 | 1 | Predose | x | x | x | x |

Abbreviations: ECG = electrocardiogram; EOT = End of Treatment; PD = pharmacodynamic; PK = pharmacokinetics; QD = once-daily.
^{a}12-lead ECG and vital sign measurements should be collected prior to any simultaneously scheduled PK, PD, or laboratory sample collections. Electrocardiogram and vital signs should be performed within 15 minutes prior to any simultaneously scheduled PK assessment. The measurement of 12-lead-ECO should be performed before vital signs if the times coincide. The 12-lead ECG assessment will be measured in duplicate and the times may be adjusted based on emerging data.
^{b}To accommodate the patient's schedule, the Day 15/16 assessments may be delayed to either the Day 22 visit or the Cycle 2 Day 1 visit.

TABLE 10
Laboratory

| assessments | Parameters | |
|---|---|---|
| Hematology | Platelet Count | White Blood Cell Count with Differential: |
| | Hemoglobin | Basophils |
| | Hematocrit | Eosinophils |
| | RBC count | Lymphocytes |
| | | Monocytes |
| | | Neutrophils (ANC) |
| | | Blast cells (if present) |
| Clinical Chemistry | AST | Magnesium |
| | ALT | Potassium |
| | ALP | Sodium |
| | GGT | Calcium |
| | LDH | Cholesterol |
| | Creatinine | Triglycerides |
| | Creatinine clearance | Uric acid |
| | Glucose (fasting) | Albumin |
| | BUN | Phosphate |
| | Amylase | Carbon dioxide (bicarbonate) |
| | Lipase | Total bilirubin |
| | Chloride | Direct bilirubin will be measured if total |
| | | bilirubin is >Grade 1 |
| Coagulation | INR | APTT |
| | Prothrombin time | |
| Urinalysis | Leucocytes | Red blood cells |
| | Protein | pH |
| | Bilirubin | Nitrite |
| | Ketones | Specific gravity |
| | Microscopy (if clinically indicated) | Glucose |
| Viral serology | HIV I and II | Anti-HCV antibody (if positive HCV-RNA |
| | HBsAg | should be performed) |
| | Anti-HBc antibody | HBV-DNA (in case the HBsAg is negative but |
| | | the anti-HBC antibody is positive, the HBV- |
| | | DNA viral load will be taken. If HBV-DNA is |
| | | undetectable, the subject may be eligible) |
| Thyroid profile | TSH | Free T4 |
| Glycosylated | HbA1c | |
| hemoglobin | | |

| | |
|---|---|
| Paraprotein^{a} | Serum M-protein by serum protein electrophoresis with immunofixation. |
| Other screening tests | Serum hCG pregnancy test (as needed for women of childbearing potential) at screening, |
| | on Day 1 of each cycle starting with Cycle 2, EOT, Follow-up Visits, and EOS. |
| | Follicle-stimulating hormone and estradiol (as needed in of non-childbearing |
| | potential only). |
| | CMV testing (serology and/or antigen) will be performed at Screening per local |
| | standard practice. Subjects with active CMV infection will not be enrolled. CMV |
| | surveillance with CMV antigen and/or POR testing will be performed as per local |
| | practice. If CMV testing is unable to be performed locally, testing will be performed by |
| | a central laboratory. |
| | The results of each test must be entered into the eCRF. |
| Other tests | Serum β2-microglobulin (biomarker for B-cell malignancies) |
| | Serum immunoglobulins: IgM, IgG, and IgA for Waldenstrom's macroglobulinemia |

Abbreviations: ALP = alkaline phosphatase; ALT = alkaline aminotransferase; ANC = absolute neutriohil count; anti-HBc = hepatitis B core antibody; AST = aspartate aminotransferase; APTT = activated partial thromboplastin time; BUN = blood urea nitrogen; CMV = Cytomegalovirus; eCRF = electronic case report form; EOT = End of Treatment; GGT = gamma-glutamyl transferase; HbA1c = hemoglobin A1c; HBsAg = hepatitis B surface antigen: HBV = hepatitis B virus; hCG = human chorionic gonadotropin; HCV = hepatitis C virus; HIV = human immunodeficiency virus; Ig = immunoglobulin; INR = International Normalized Ratio; LDH = lactate dehydrogenase; PCR = polymerase chain reaction; RBC = red blood cell; T4 = thyroxine; TSH = thyroid stimulating hormone.
^{a}Subjects with Waldenstrom macroglobulinemia only.

All laboratory tests with values considered clinically significantly abnormal during participation in the clinical trial or within 30 days after the last dose of test drug treatment should be repeated until the values return to normal or baseline or are no longer considered clinically significant by the investigator or medical monitor. On the day of administration of the test drug, samples for these parameters are collected prior to dose administration. Additional evaluations may be performed at the investigator's discretion if medically indicated. In addition, laboratory assessments may be performed if a subject has provided a paired biopsy tumor sample for analysis of biomarkers.

At screening, a serum pregnancy test must be performed within 3 days before the first dose. During the study (on Day 1 of each cycle starting with Cycle 2), EOT, Follow-up Visits, and EOS a serum pregnancy test must be performed. If a test subject (female) becomes pregnant, administration of the test drug needs to be stopped immediately. If a pregnancy test is positive, but the subject is thought not to be pregnant, the test drug administration should be delayed until it is determined that the test was falsely positive and pregnancy is excluded.

2) Adverse Events (1) Definition of Adverse Event and Serious Adverse Event

An adverse event (AE), as defined in this clinical trial, is any untoward medical occurrence in a subject, temporally associated with the use of test therapy, whether or not considered related to the test therapy (Table 11).

TABLE 11
AE Definition
An AE is any untoward medical occurrence in a subject of subject, temporally associated with the use
of study treatment, whether or not considered related to the study treatment.
NOTE: An AE can therefore be any unfavorable and unintended sign (including an abnormal
laboratory finding). symptom, or disease (new or exacerbated) temporally associated with the use of
study treatment.
Events Meeting the AE Definition
Any abnormal laboratory test results (hematology, clinical chemistry, or urinalysis) or other safety
assessments (eg, ECG, radiological scans, or vital signs measurements), including those that worsen
from baseline, considered clinically significant in the medical and scientific judgment of the
Investigator (ie, not related to the progression of underlying disease).
Exacerbation of a chronic or intermittent preexisting condition including either an increase in
frequency and/or severity of the condition.
New conditions detected or diagnosed after study treatment administration even though it may have
been present before the start of the study.
Signs, symptoms, or the clinical sequelae of a suspected drug-drug interaction.
Signs, symptoms, or the clinical sequelae of a suspected overdose of either study treatment or a
concomitant medication. Overdose per se will not be reported as an AE/SAE unless it is an intentional
overdose taken with possible suicidal/self-harming intent. Such overdoses should be reported
regardless of sequelae.
“Lack of efficacy” or “failure of expected pharmacological action” per se will not be reported as an AE
or SAE. Such instances will be captured in the efficacy assessments. However, the signs, symptoms,
and/or clinical sequelae resulting from lack of efficacy will be reported as AE or SAE if they fulfill the
definition of an AE or SAE.
Events NOT Meeting the AE Definition
Any clinically significant abnormal laboratory findings or other abnormal safety assessments which are
associated with the underlying disease, unless judged by the Investigator to be more severe than
expected for the subject's condition
The disease/disorder being studied or expected progression, signs or symptoms of the disease/disorder
being studied, unless more severe than expected for the subject's condition.
Medical or surgical procedure (eg, endoscopy and appendectomy): the condition that leads to the
procedure is the AE.
Situations in winch an untoward medical occurrence did not occur (social and/or convenience
admission to a hospital).
Anticipated day-to-day fluctuations of preexisting disease(s) or condition(s) present or detected at the
start of the study that do not worsen.

In this clinical trial, if an event does not correspond to the above-defined adverse events, then it cannot be a serious adverse event (SAE) even if serious conditions are met (e.g., hospitalization for signs/symptoms of the disease under clinical trial or death due to progression of disease). The definition of serious adverse event is summarized below (Table 12).

TABLE 12
An SAE is defined as any untoward medical occurrence that, at any dose:

a) Results in death
b) Is life-threatening
The term ‘life-threatening’ in the definition of “serious” refers to an event in which the subject was at
risk of death at the time of the event. It does not refer to an event, which hypothetically might have
caused death, if it were more severe.
c) Requires inpatient hospitalization or prolongation of existing hospitalization
In general, hospitalization signifies that the subject has been detained (usually involving at least an
overnight stay) at the hospital or emergency ward for observation and/or treatment that would not have
been appropriate in the physician's office or outpatient setting. Complications that occur during
hospitalization are AEs. If a complication prolongs hospitalization or fulfills any other serious criteria,
the event is serious. When in doubt as to whether “hospitalization” occurred or was necessary, the AE
should be considered serious.
Hospitalization for elective treatment of a preexisting condition that did not worsen from baseline is not
considered an AE.
d) Results in persistent disability/incapacity
The term disability means a substantial disruption of a person's ability to conduct normal life
functions.
This definition is not intended to include experiences of relatively minor medical significance such as
uncomplicated headache, nausea, vomiting, diarrhea, influenza, and accidental trauma (eg, sprained
ankle) which may interfere with or prevent everyday life functions but do not constitute a substantial
disruption.
e) Is a congenital anomaly/birth defect
f) Other situations:
Medical or scientific judgment should be exercised in deciding whether SAE reporting is appropriate
in other situations such as important medical events that may not be immediately life-threatening of
result in death or hospitalization but may jeopardize the subject or may require medical or surgical
intervention to prevent 1 of the other outcomes listed in the above definition. These events should
usually be considered serious.
Examples of such events include invasive or malignant cancers, intensive treatment in an emergency
room or at home for allergic bronchospasm, blood dyserasias or convulsions that do not result in
hospitalization, or development of drug dependency of drug abuse.

In addition, the severity assessment criteria of this clinical trial are summarized below (Table 13).

TABLE 13
Assessment of Severity

The Investigator will make an assessment of severity for each AE or SAE reported during the study and
assign it to 1 of the following categories based on the National Cancer Institute (NCI) CTCAE version 5.0,
a descriptive terminology that can be used for AE reporting:
A general grading (referred to as severity) scale is provided at the beginning of the above referenced
document, and specific event grades are also provided.
If the severity for an AE is not specifically graded by NCI-CTCAE, the Investigator is to use the general
NCI-CTCAE definitions of Grade 1 through Grade 5, using his or her best medical judgment.
The 5 general grades are:
Grade 1 or Mild
Grade 2 or Moderate
Grade 3 or Severe
Grade 4 or Life-threatening
Grade 5 or Death
An event is defined as ‘serious’ when it meets at least 1 of the predefined outcomes as described in the
definition of an SAE, NOT when it is rated as severe, eg. a laboratory abnormality of Grade 4, such as
anemia or neutropenia, is considered serious only if the condition meets 1 of the criteria specified in the
definition of an SAE.
If death occurs, the primary cause of death or event leading to death should be recorded and reported as an
SAE. “Fatal” will be recorded as the outcome of this specific event and death will not be recorded as
separate event. Only, if no cause of death can be reported (eg, sudden or unexplained death), the death per
se might then be reported as an SAE.

All adverse events (AEs) and serious adverse events (SAEs) are collected from the signing of the informed consent form (ICF) until the 30-day Safety Follow-up at the time points specified in the schedule of activities.
Care needs to be taken not to introduce bias when detecting AEs and/or SAEs. And Open-ended and non-leading verbal questioning of the subjects is the preferred method to inquire about AE occurrences.
Details of all pregnancies in female subjects and female partners of male subjects are collected after the start of test drug administration and until 3 months after the last administration of test drug. Abnormal pregnancy outcomes (e.g., spontaneous abortion, fetal death, stillbirth, congenital anomalies, and ectopic pregnancy) are considered SAEs.

### F. Efficacy Assessment

1) Tumor response is assessed for both phases la and Ib. The anticancer activity of the test drug is assessed by applying various criteria through imaging tests every two cycles of clinical trials and at the end of therapy (EOT) (optional) (see table 14 for disease assessment criteria below), or for liquid tumors, bone marrow aspirates/biopsies and blood assessments are performed. Tumor response parameters that are assessed include the following:

(1) Best overall response: complete response (CR), partial response (PR), stable disease (SD, lasting 12 weeks), or disease progression (DP)

(2) Objective response rate (ORR): complete response (CR)+partial response (PR)

(3) Clinical benefit rate (CBR): complete response (CR) or partial response (PR) or stable disease (SD) lasting at least 12 weeks

(4) Duration of response or duration of SD

(5) Time-to-event endpoints of time to progression (TTP), progression-free survival (PFS), and overall survival (OS) during the test observation period are also assessed.

2) Disease assessment criteria

Details of the disease assessment criteria and the tools used to determine efficacy parameters are as follows (Table 14).

TABLE 14

| Group: population | Tool to define: CR, PR, SD, and disease progression |
|---|---|
| A: Patients (subjects) with DLBCL, including | Lugano Criteria^{a} |
| MYC-altered DLBCL | |
| B: Patients (subjects) with follicular lymphoma | Lugano Criteria^{a} |
| C: Patients (subjects) with CLL/SLL | Hallek CLL International Workshop Guidelines^{b} |
| C: Patients (subjects) with PTCL or with other B-cell | Lugano Criteria^{a} |
| lymphoma such as, but not limited to: mantle cell | |
| lymphoma and marginal zone lymphoma | |
| C: Patients (subjects)with Waldenstrom macroglobulinemia | Owen et al.^{c} |

Abbreviations: CLL = chronic lymphocytic leukemia; CR = complete response; DLBCL = diffise large B-cell lymphoma; PTCL = peripheral T cell lymphoma; PR = partial response; SD = stable disease; SLL = small lymphocytic leukemia.
^{a}Lugano criteria: Criteria for response assessment
^{b}Halleck CLL guidelines are response definition after Treatment of Chronic Lymphocytic Leukemia patient (subject)
^{c}Waldenstrom macroplobulinaemia criteria is response assessment in Waldenstrom Macroglobulinaemia

3) Baseline Tumor Assessments

At screening, potential subjects with lymphoma undergo a positron emission tomography (PET) scan with 2 deoxy-2-[fluoro-18]fluoro-D-glucose along with a PET-CT scan. The scan should be performed a minimum of 3 to a maximum of 10 days prior to blood sampling to avoid radioactive contamination of the blood sample.

At screening, a diagnostic quality CT scan with IV contrast of chest/abdomen/pelvis/additional known lesions is performed for all subjects (for those subjects who are imaged with PET-CT, the imaging studies may be combined). If at screening, a subject has a medical contraindication to the CT IV contrast or develops a contraindication during the clinical trial, the following radiologic assessments are performed: a non-contrast chest CT and a contrast abdomen/pelvis magnetic resonance imaging (MRI). Chest x-rays and ultrasound should not be used to measure tumor lesions.

If available and of acceptable quality (e.g., CT portion of PET/CT scan was performed with contrast), a previously performed PET/CT scans that were done within 21 days prior to start of treatment may be used for baseline radiology assessment.

All subsequent disease assessments are performed with CT scan with IV contrast. For subjects staged with CT, up to 6 of the largest target nodes, nodal masses, or other lymphomatous lesions that are measurable in 2 diameters (longest diameter [LDi] and shortest diameter) should be identified from different body regions representative of the subject's overall disease burden and include mediastinal and retroperitoneal disease, if involved. A measurable node must have an LDi greater than 1.5 cm. Measurable extranodal disease may be included in the 6 representative, target lesions. A measurable extranodal lesion should have an LDi greater than 1.0 cm. All other lesions (including nodal, extranodal, and assessable disease) should be followed as non-target disease.

Brain MRI or CT needs to be completed at screening only if clinically indicated. Contrast-enhanced brain MRI is preferred. However, if MRI contrast is contraindicated, then MRI without contrast or CT with/without contrast is acceptable.

4) Baseline Tumor Assessment

Each lesion that is measured at baseline must be remeasured by the same method (either radiologic or nuclear method) throughout the clinical trial so that the comparison is consistent. For CT scans there is a ±7 days window for the scheduled assessment. Subjects with PET-avid disease will have a repeat PET-CT to verify complete response (CR). The PET-CT scans should be performed a minimum of 3 to a maximum of 10 days prior to blood sampling to avoid radioactive contamination of the blood sample. Additional imaging assessments may be performed at any time during the clinical trial at the investigator's discretion to support the efficacy evaluations for a subject, as necessary. Clinical suspicion of disease progression at any time requires a physical examination and imaging assessments to be performed promptly rather than waiting for the next scheduled imaging assessment. If an off-schedule imaging assessment is performed because progression is suspected, subsequent imaging assessments should be performed in accordance with the original imaging schedule.

For CR, after consultation with the medical monitor, a CR confirmatory PET or PET-CT may be performed. The PET-CT scan should be performed a minimum of 3 to a maximum of 10 days prior to blood sampling. In lymphoma subjects with bone marrow involvement prior to the clinical trial start a bone marrow biopsy should be performed to confirm CR.

### G. Population for Analysis

The definition of an analysis group is as follows (Table 15).

TABLE 15

| Analysis set | Description |
|---|---|
| Entered Analysis Set | All subjects who sign the ICF. |
| Enrolled Analysis Set | All subjects who have met all eligibility criteria. |
| Full Analysis Set | The Full Analysis Set comprises all subjects who receive at least 1 dose of test |
| | drug and have at least 1 valid postbaseline tumor evaluation. |
| Safety Analysis Set | All subjects who take at least 1 dose of test drug. Subjects will be analyzed |
| | according to the dose and regimen they actually received. |
| DLT Evaluable Set | The DLT Evaluable Set analysis set consists of all subjects from the Safety |
| | Analysis Ser in Phase Ia (dose escalation) who have received at least 1 dose of test |
| | drug during Cycle 1. These constitute the evaluable subjects for the determination |
| | of MTD. |
| PK Analysis Set | All subjects in the Safety Analysis Set who have at least 1 quantifiable plasma |
| | and/or urine test drug concentration collected postdose without important protocol |
| | deviations/violations or events thought to significantly affect the PK. |
| PD Analysis Set | All subjects in the Safety Analysis Set that have at least 1 PD variable collected |
| | postdose without important protocol deviations/violations of events thought to |
| | significantly affect the PD. |

Abbreviations: DLT = dose limiting toxicity; ICF = informed consent form; MTD = maximum tolerated dose; PD = phamicodynamic; PK = phannacokinetic.

### 5. Statistical Method

### A. Efficiency Analysis

The tumor response (lymphoma (B-cell and T-cell), CLL/SLL, Waldenstrom macroglobulinemia) following administration of the test drug in phases Ia and Ib is assessed using the criteria in table 14. Tumor response parameters vary according to the above applied criteria and may include the followings:

1) Best Overall Response: Complete Response (CR), Partial Response (PR), Stable Disease (SD) or Disease Progression (DP)

The best overall response is defined as the best response across all time points (in the order of CR, PR, SD, and DP) post-treatment. The best overall response (CR, PR, SD, or DP) is summarized by number of subjects and percentages. In the case of best response equal to SD, the minimum duration for SD should be met. For the purpose of calculating an SD rate, the rate will be the percentage (%) of subjects who continue to have SD for at least 12 weeks.

2) Objective Response Rate (ORR): CR+PR

The objective response rate (ORR) is the proportion of subjects with a best overall response of complete response (CR) or partial response (PR). The ORR and its 95% confidence interval (CI) are presented. The 95% (exact) CI is calculated based on the Clopper-Pearson method.

3) Clinical Benefit Rate (CBR): CR or PR or SD Lasting at Least 12 Weeks

Clinical benefit rate (CBR) is defined as the proportion of subjects who achieve complete response (CR), partial response (PR), or durable stable disease (SD) (SD≥12 weeks) based on the Lugano revised criteria for response assessment. The CBR as well as 95% (exact) CI are estimated by the Clopper-Pearson method.

4) Duration of Response

Duration of response for responders (CR or PR) is defined as the time interval between the date of the earliest qualifying response and the date of disease progression or death for any cause, whichever occurs earlier. For subjects who are alive without disease progression following the qualifying response, duration of response is censored on the date of last evaluable tumor assessment or last Follow-up Visit for disease progression. Duration of response is summarized in subjects with a complete response (CR) or partial response (PR) using the Kaplan-Meier method. Median event time, quartiles Q1, Q3 and 2-sided 95% CI for the median is provided using the Brookmeyer-Crowley method.

5) Duration of SD

Duration of stable disease (SD) is defined as the time interval, in the absence of either complete response (CR) or partial response (PR) between the date of the first-time achieving SD after administration of test drug and the date of disease progression or death for any cause, whichever occurs earlier. For subjects who are alive without disease progression, duration of SD is censored on the date of last evaluable tumor assessment. Duration is summarized in subjects with a SD using the Kaplan-Meier method. The median event time, Q1, Q3, and 2-sided 95% CI for the median is provided using the Brookmeyer-Crowley method.

6) Time to Tumor Progression (TTP)

Time to tumor progression (TTP) is defined as the time from the date of first study dose to disease progression. Subjects without disease progression are censored at the date of ‘last evaluable tumor assessment’ or date of first dose, whichever comes later. If no postbaseline assessment is available, TTP is to be censored at the date of first dose. Time to progression is presented graphically as Kaplan-Meier curves. The median event time, Q1, Q3 and 2-sided 95% CI for the median is provided using the Brookmeyer-Crowley method.

7) Progression-Free Survival

Progression-free survival (PFS) is defined as the time from the date of first study dose to disease progression or death whichever occurs first in subjects. Subjects without event (no disease progression or death) are censored at the date of ‘last evaluable tumor assessment’. Subjects for whom no postbaseline evaluable tumor assessments are available are censored at the time of first dose. Progression-free survival is summarized in the same way as tumor progression (TTP).

8) Overall Survival During Study Observation Period

Overall survival (OS) is defined as the time from the date of first dose until the date of death from any cause. For subject who is not known to have died by the EOS follow-up, OS is censored at the date of last contact. Date of last contact is defined as the latest of the following dates:

Treatment discontinuation date, last dosing administration date, last disease assessment date or the last follow-up date on which the subject was known to be alive. Overall survival will be presented graphically as Kaplan-Meier curves. Overall survival will be summarized in the same way as progression-free survival (PFS).

### B. Safety Analysis

1) Phase Ia (Dose Escalation)

The number and proportion of subjects experiencing the dose limiting toxicity (DLT) are summarized based on the dose-limiting toxicity evaluable set. The corresponding 95% exact CI according to the Clopper-Pearson method is provided. Estimation of maximum tolerated dose (MTD)/recommended dose for clinical phase Ib/II (RP2D) are based upon the methods for determination of MTD/RP2D provided herein (see 3. Overall design, 2) How to escalate dose of test drug).

2) Phase Ia (Dose Escalation) and Phase Ib (Dose Expansion)

The following analyses are performed on the safety analysis set.

(1) Treatment emergent adverse event (TEAE):

A treatment emergent adverse event (TEAE) is defined as any AE that occurs after the first administration of the test drug through 30 days after the last administration of the test drug, or any event that is present at baseline but worsens in severity. The TEAE are to be coded and grouped using the Medical Dictionary for Regulatory Activities (MedDRA) Version 21.1 or higher. All the adverse events and toxicities are graded according to CTCAE (version 5.0). Analysis may include, but is not limited to, the followings:

TEAEs, treatment-related TEAEs, Grade 3 or higher TEAEs, Grade 3 or higher treatment-related TEAEs, TEAEs resulting in discontinuation of test drug, serious TEAEs, serious treatment-related TEAEs, and TEAEs leading to death. The number and percent of subjects are summarized for the above items, and also summarized according to MedDRA System Organ Class (SOC) and Preferred Term (PT). A 2-sided 95% CI for the percentage of subjects in each TEAE category is provided. The severity and causality of the TEAE are summarized. All AEs occurring on the test are presented in data listing and the TEAEs are flagged.

Additional summaries are also provided using adverse events of special interest. The adverse events of special interest may include, but are not limited to, the followings:

Hepatotoxicity, skin rash, diarrhea/colitis, pneumonitis, decrease leukocytes, anemia, increased rates of infection including opportunistic infections such as PJP and CMV infection.

(2) Clinical laboratory parameters (hematology, clinical chemistry, coagulation, thyroid, glycosylated hemoglobin [HbA1c] and urinalysis) are summarized at each scheduled time. Changes over time are described using descriptive statistics, and the percentage of subjects with values outside the normal range is presented. In addition to the high and low laboratory assignments, clinically significant laboratory assessments are also identified. Incidence of abnormal values according to clinically significant laboratory evaluation results are provided. Clinical laboratory data is graded according to CTCAE. For laboratory tests where grades are not defined by CTCAE, results are graded by the low/normal/high classifications based on laboratory normal ranges. Frequency tables are used for newly occurring on-treatment Grades 3 or 4. Shift tables are produced for the comparison of baseline and postbaseline values at cach sample time point for the selected laboratory parameters. Descriptive statistics are used to summarize vital sign results (pulse rate, respiration rate, body temperature, systolic and diastolic blood pressure) and changes from baseline. Data from the ECG central reviewer are used in summary presentations. The 2 values of each ECG parameter within a time point from the central reviewer are averaged to determine time specific parameter for a subject and used in summaries. Electrocardiogram variables are summarized using descriptive statistics at each scheduled time point, including change from baseline. Number and percentage of subjects with normal and abnormal results as assessed by the central reviewer for the overall interpretation are tabulated for each time point. Shift tables from baseline to worst postbaseline are produced.

(3) ECOG performance status is summarized by each visit.

(4) The physical examination is listed only. All clinically significant abnormal findings are recorded as medical history or adverse events.

(5) The results of pregnancy tests for females are listed.

(6) Number of cycles administered, administered dose, cumulative dose, dose intensity, relative dose intensity, and the overall duration of exposure are summarized for the Safety Analysis Set (SAS) and are summarized using standard summary statistics. The total dose per cycle for each subject is summarized using descriptive statistics. Tolerability of the test drug is assessed by summarizing the number of dose delays, interruptions, and dose reductions. Reasons for dose delays, interruptions, and dose reductions are listed by subject and summarized. The reasons for discontinuation from treatment are summarized and listed, along with dates of first and last administration of the test drug, the duration of exposure to the test drug, and the date of discontinuation of administration.

(7) All pre-medication and concomitant drug data are listed. Prior medications are those medications that are stopped prior to first dose of clinical trial treatment. Concomitant medications are medications taken at least once on or after first dose of clinical trial treatment. Medications started prior to and stopped on the same day as first dose of study treatment are considered as prior medication only. All medications are coded using the most recent version of the WHO Drug Dictionary/WHO Drug Reference List. The number and percentage of subjects is presented by PT and Anatomical Therapeutic Chemical Classification System level 1 and level 2 for prior and concomitant medications. All prior and concomitant medication data is listed.

(8) Non-medication therapy name is coded using MedDRA. The number and percentage of subjects is presented by PT. Non-medication therapy information is listed.

(9) Demographics, baseline medical status, medical history, and other baseline characteristics are summarized and listed for full analysis set (FAS).

### C. Pharmacokinetic (PK) Analysis

Plasma concentrations and PK parameters of the test drug are listed and summarized by dose, cycle, and clinical trial phase using descriptive statistics.

Urine concentrations and parameters of the test drug are listed and summarized for each dose using descriptive statistics. The plasma concentration-time profile of the test drug is displayed graphically.

Scatter plots of individual values and geometric means of PK exposure parameters, as applicable, including area under the plasma concentration-time curve from time zero during a dosing interval [AUC₍₀₋ₜₐᵤ₎], area under the plasma concentration-time curve from time zero extrapolated to infinity [AUC_{(0-inf)}], or area under the plasma concentration-time curve from time zero to the time of the last quantifiable concentration [AUC₍₀₋ₗₐₛₜ₎] if AUC_{(0-inf)} is not calculable in a majority of subjects, and Cₘₐₓ versus dose will be presented by study day. Dose proportionality of PK exposure parameters Cₘₐₓ, AUC₍₀₋ₜₐᵤ₎, and AUC_{(0-inf)} [or AUC₍₀₋ₗₐₛₜ₎: if AUC_{(0-inf)} is not calculable in in most subjects] over the administered dose range are investigated using a power model. Time linearity and test drug accumulation may be evaluated by using an analysis of variance model. Effect of sex, age, and/or body weight may be explored graphically or by utilizing inferential analyses. Further details on any planned statistical analyses are provided in the Statistical Analysis Plan (SAP). Attainment of steady-state conditions are visually evaluated by graphical display of predose the test drug concentrations.

### D. Pharmacodynamic (PD) Analysis

Exploratory PD and predictive biomarker data are listed and, if appropriate, presented in descriptive summaries for observed and change from baseline values.

### E. Pharmacokinetic (PK)-Pharmacodynamic (PD) Analysis

Relationship between test drug exposure and tumor size reduction may be explored graphically by means of scatter plots.

### F. Interim Analysis

Details of the interim analyses to be conducted are described in a statistical analysis plan (SAP).

1) Phase Ia (dose escalation): Two interim analyses are done during the Phase Ia trial as follows.
After completion of cycle 1 of the last subject in cohort 3.
After completion of cycle 1 of the last cohort in phase Ia.

2) Phase Ib (dose expansion): One interim analysis is performed when all subjects in phase 1b have been treated or followed for enough time to have completed at least 6 cycles of treatment or have been followed to treatment discontinuation if less than 6 cycles.

### 6. Assessments Results

### A. Safety Assessment Results

It was confirmed that 2 of 11 clinical trial subjects showed grade 3 skin reaction and 3 clinical trial subjects showed hepatotoxicity. All of the above adverse events were manageable and recovered to grade 0 or 1 upon discontinuation of the test drug.

The safety assessment results for 5 clinical trial subjects (DLBCL (n=1) and PTCL (n=4)) included in cohorts 1 to 3 are as follows:

Treatment-emergent adverse events (TEAEs) occurred in 4 out of 5 (80.0%) subjects. Of those events, the TEAEs in 3 subjects were evaluated to be related to the test drug (60.0%, 3 cases), and no administration-related deaths occurred. 2 subjects showed TEAE of grade 2 (moderate), and respectively 1 subject showed the TEAE of grade 1 (mild) and grade 4 (life-threatening). The grade 4 TEAE was thrombocytopenia and was not related to the test drug.

According to the above results, no serious adverse events rated as grades 3 (severe) to 5 (death) occurred in cohorts 1 to 3, and thus it was confirmed that there was no dose limiting toxicity (DLT) in the dose range of cohorts 1 to 3.

The safety assessment results for cohort 4 are as follows:

In cohort 4 (325 mg QD), two dose limiting toxicities (DLTs) were observed. Accordingly, it was considered that the maximum tolerated dose (MTD) was exceeded, and the expansion of the dosing to the cohort was terminated. The administered dose was de-escalated to the previous lower dose level (200 mg QD) and extended to enrollment of 3 additional subjects for the de-escalated dose. No dose limiting toxicity was confirmed in 6 subjects in the expansion cohort (200 mg QD).

From the safety assessment results for the above cohorts 1 to 4, it was confirmed that the maximum tolerated dose (MTD) was 200 mg QD, and it was confirmed that the tolerability was excellent in this dose range. In addition, 200 mg QD was recommended as the starting dose (RP2D) for clinical phase Ib/II by the Safety Monitoring Committee.

### B. Efficacy Assessment Results . . . Tumor Response Assessment

Tumor response assessment was confirmed in 11 of 12 subjects enrolled in clinical phase Ia (dose escalation part). Specifically, a complete response (CR) in 1 subject and a partial response (PR) in 2 subjects were confirmed, and stable disease (SD) were observed in 6 subjects. In addition, disease progression (PD) was confirmed in 2 subjects.

Among 12 enrolled subjects, in particular, in the assessment of tumor response of 8 subjects diagnosed with peripheral T-cell lymphoma (PTCL), 1 subject with complete response (CR), 2 subject with partial responses (PR), 4 subject with stable diseases (SD) and 1 subject with disease progression (PD) were observed, and a disease control rate (DCR) was 87.5%.

From the above results, it was confirmed that the test drug showed preliminary activity in peripheral T-cell lymphoma (PTCL) subjects.

### C. Pharmacokinetic and Pharmacodynamic Assessment Results

1) Pharmacokinetics

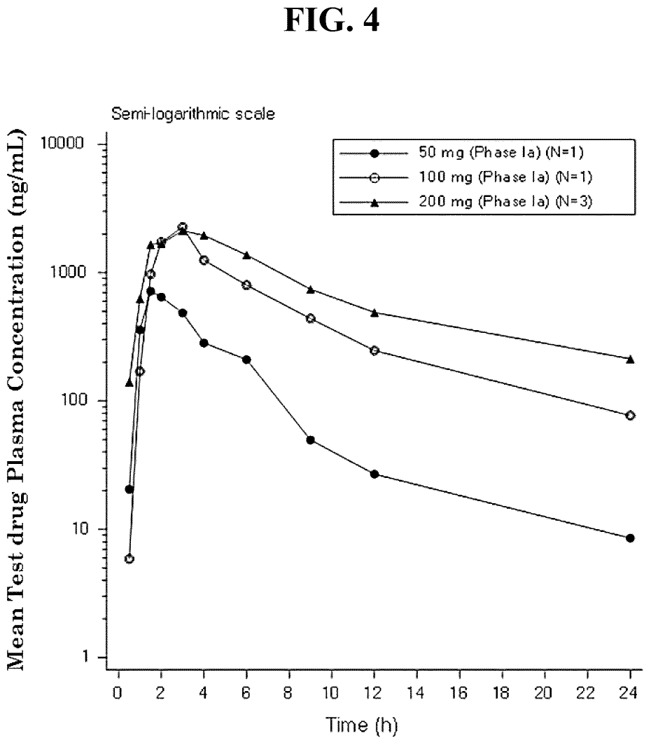

On the day 1 of cycle 1 (C1D1), plasma samples for pharmacokinetic analyses in clinical trial subjects were obtained before and after administration of the test drug (0.5, 1, 1.5, 2, 3, 4, 6, 9, 12 and 24 hours). As a result of the collected pharmacokinetic analyses, it was confirmed that a maximum plasma concentration after a single dose of 50 mg QD, 100 mg QD and 200 mg QD of the test drug ranged from to 2443 ng/ml, and a systemic exposure (based on AUC₀₋₂₄) ranged from 2760 to 21850 ng*h per mL. It was confirmed that the exposure concentration of the test drug increased in a dose-dependent manner in the dose range of 50 mg to 200 mg (FIG. 4).

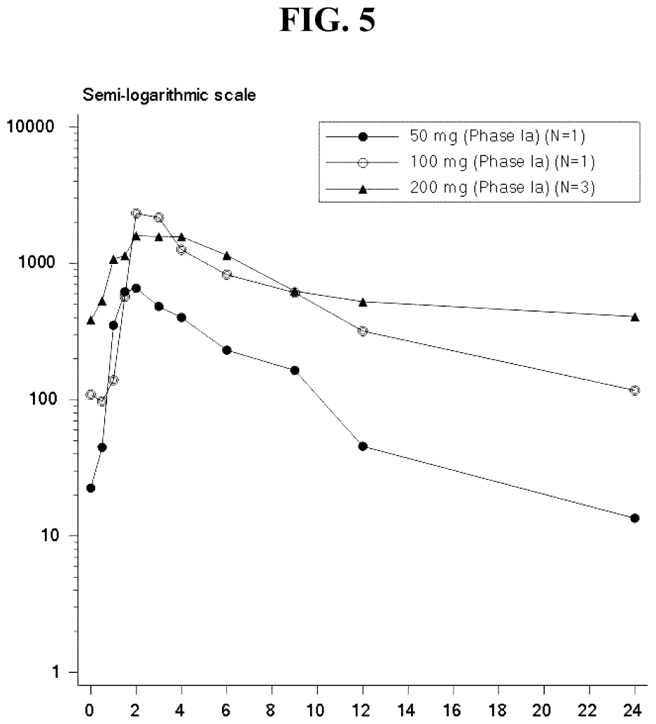

In addition, the mean plasma concentration values in the multiple dosing range of the 50 mg QD, 100 mg QD, and 200 mg QD cohorts showed the PK values from multiple dosing range of 50 mg to 200 mg cohort resulted in an approximate 2.92-fold and 4.97-fold increase in exposure based on the Cₘₐₓ and AUCₜₐᵤ, respectively. In addition, it was confirmed that the exposure concentration of the test drug increased in a dose-dependent manner in the dose range of 50 mg to 200 mg, and there was no accumulation observed in the dose range of 50 mg to 200 mg. (FIG. 5).

2) Pharmacodynamics

Blood samples for pharmacodynamic analyses of the test drug were collected in phase Ia (dose escalation part). Whole blood samples were evaluated by flow-cytometry after coimmunostaining for CD3⁺, CD4⁺, CD25⁺, CD127low and CD8⁺. Through this, we tried to observe the effects of the test drug on regulatory T cells (T_{regs}) and CD8⁺ T cells.

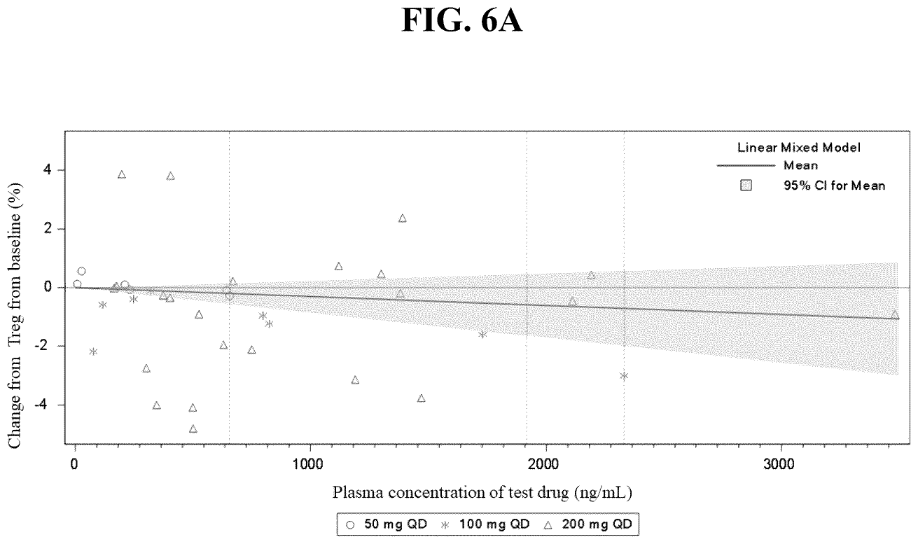

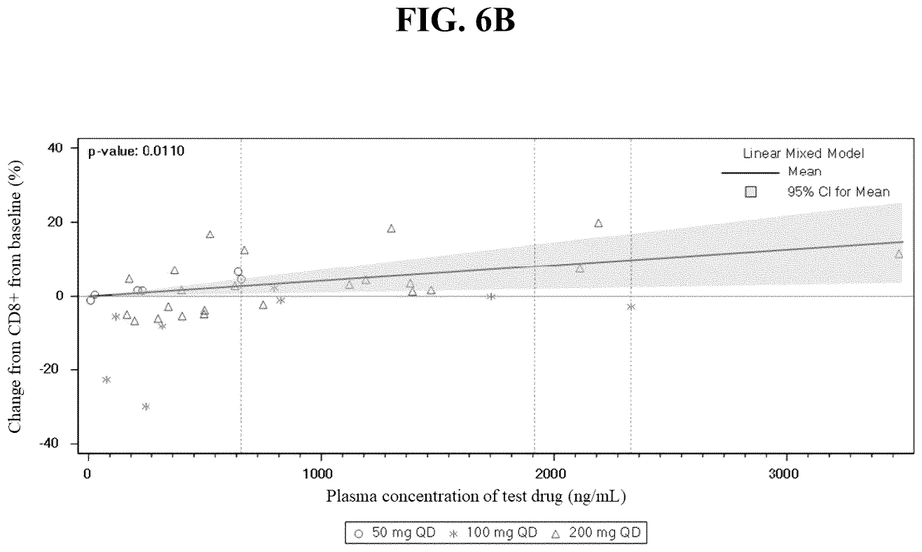

As a result, it was confirmed that as the plasma concentration of the test drug increased, regulatory T cells (T_{regs}) decreased and CD8⁺ T cells increased compared to the baseline value (FIGS. 6A and 6B). The above results confirm that the decrease in regulatory T cells (T_{regs}) and the increase in CD8⁺ T cells occur depending on the exposure of the test drug according to the pharmacokinetics, thereby confirming that the pharmacokinetics of the test drug and the pharmacodynamics are correlated with each other.

What is claimed is:

1. A pharmaceutical composition for preventing or treating peripheral T-cell lymphoma (PTCL), comprising a compound represented by formula 1 below or pharmaceutically acceptable salts thereof in an amount of 25 mg or more and less than 325 mg as a compound represented by formula 1:

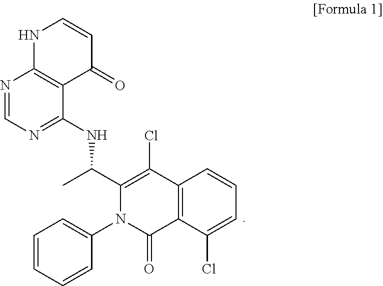 [SMILES: C[C@H](Nc1ncnc2[nH]ccc(=O)c12)c1c(Cl)c2cccc(Cl)c2c(=O)n1-c1ccccc1]

2. The pharmaceutical composition according to claim 1, wherein the composition comprises the compound represented by above formula 1 or pharmaceutically acceptable salts thereof in an amount of 200 mg or more and less than 325 mg as the compound represented by formula 1.

3. The pharmaceutical composition according to claim 1, wherein the composition comprises the compound represented by above formula 1 or pharmaceutically acceptable salts thereof in an amount of 200 mg as the compound represented by formula 1.

4. The pharmaceutical composition according to claim 1, wherein the composition is administered to a subject who has received at least one or more chemotherapies after being diagnosed with peripheral T-cell lymphoma.

5. The pharmaceutical composition according to claim 1, wherein the composition is administered once daily for four weeks or more.

6. The pharmaceutical composition according to claim 1, wherein the composition is orally administered.

7. The pharmaceutical composition according to claim 1, wherein the composition decreases regulatory T cells (T_{regs}) and increases CD8⁺ T cells.

8. The pharmaceutical composition according to claim 2, wherein the composition is administered once daily for four weeks or more.

9. The pharmaceutical composition according to claim 2, wherein the composition is orally administered.

10. The pharmaceutical composition according to claim 2, wherein the composition decreases regulatory T cells (T_{regs}) and increases CD8⁺ T cells.

11. The pharmaceutical composition according to claim 3, wherein the composition is administered once daily for four weeks or more.

12. The pharmaceutical composition according to claim 3, wherein the composition is orally administered.

13. The pharmaceutical composition according to claim 3, wherein the composition decreases regulatory T cells (T_{regs}) and increases CD8⁺ T cells.

14. The pharmaceutical composition according to claim 4, wherein the composition is administered once daily for four weeks or more.

15. The pharmaceutical composition according to claim 4, wherein the composition is orally administered.

16. The pharmaceutical composition according to claim 4, wherein the composition decreases regulatory T cells (T_{regs}) and increases CD8⁺ T cells.
